(19) United States
(12) Patent Application Publication
(10) Pub. No.: US 2002/0164333 A1
(43) Pub. Date: Nov. 7, 2002

(54) Bifunctional molecules and vectors complexed therewith for targeted gene delivery

(75) Inventors: Glen R. Nemerow, Encinitas, CA (US); Erguang Li, San Diego, CA (US)

Correspondence Address: STEPHANIE SEIDMAN, HELLER EHRMAN WHITE & MCAULIFFE LLP, 4350 LA JOLLA VILLAGE DRIVE, 7th FL., SAN DIEGO, CA, 92122-1246, US

(73) Assignee: The Scripps Research Institute

(21) Appl. No.: 09/903,327

(22) Filed: Jul. 10, 2001

Related U.S. Application Data
(60) Provisional application No. 60/325,781, filed on Jul. 10, 2000.

(51) Int. Cl. A61K 39/395; C07K 16/40

(52) U.S. Cl. 424/146.1; 530/388.26; 530/391.1

(57) ABSTRACT

Methods and products for targeting delivery vectors, such as adenoviral gene delivery particles, to selected cell types are provided. The methods rely on targeting by a bifunctional molecule that specifically complexes with a protein on the vector particle surface and with targeted cell surface proteins. The targeted cell surface proteins are any that activate the phosphatidylinositol-3-OH kinases. The bifunctional molecules, compositions, kits, and methods of preparation and use of the vector/bifunctional molecules for gene therapy are provided.

### RELATED APPLICATIONS

Benefit of priority under 35 U.S.C. §119(e) to U.S. application Ser. No. 09/613,017, entitled “BIFUNCTIONAL MOLECULES AND VECTORS COMPLEXED THEREWITH FOR TARGETED GENE DELIVERY”, by Glen R. Nemerow and Erguang Li, filed Jul. 10, 2000, and converted to a provisional application on Jul. 10, 2001.

Work described herein was supported by NIH grants EY11431 and HL54352. The government has certain rights in such subject matter.

### FIELD OF INVENTION

The present invention relates to gene therapy, especially to adenovirus vector-based gene therapy. In particular, adenovirus vectors complexed with bifunctional molecules for targeted delivery of therapeutic and other products are provided. The bifunctional molecules complexed with adenovirus delivery particles circumvent Coxsackie Adenovirus Receptor (CAR) and integrin interactions and improve gene delivery by such particles. The bifunctional molecules, compositions, kits, and methods of preparation and use of the vector/bifunctional molecules for gene therapy are provided.

### BACKGROUND OF THE INVENTION

Adenovirus Delivery Vectors

Adenovirus, which is a DNA virus with a 36 kilobase (kb) genome, is very well-characterized and its genetics and genetic organization are understood. The genetic organization of adenoviruses permits substitution of large fragments of viral DNA with foreign DNA. In addition, recombinant adenoviruses are structurally stable and no rearranged viruses are observed after extensive amplification.

Adenoviruses have been employed as delivery vehicles for introducing desired genes into eukaryotic cells. The adenovirus delivers such genes to eukaryotic cells by binding to cellular receptors followed by internalization. The adenovirus fiber protein is responsible for binding to cells. The fiber protein has two domains, a rod-like shaft portion and a globular head portion that contains the receptor binding region. The fiber spike is a homotrimer, and there are 12 spikes per virion. Human adenoviruses bind to and infect a broad range of cultured cell lines and primary tissues from different species.

The 35,000+base pair (bp) genome of adenovirus type 2 has been sequenced and the predicted amino acid sequences of the major coat proteins (hexon, fiber and penton base) have been described (see, e.g., Neumann et al., Gene 69: 153-157 (1988); Herisse et al., Nuc. Acids Res. 9: 4023-4041 (1981); Roberts et al., J. Biol. Chem. 259: 13968-13975 (1984); Kinloch et al., J. Biol. Chem. 259: 6431-6436 (1984); and Chroboczek et al., Virol. 161: 549-554, 1987).

The 35,935 bp sequence of Ad5 DNA is also known and portions of many other adenovirus genomes have been sequenced. The upper packaging limit for adenovirus virions is about 105% of the wild-type genome length (see, e.g., Bett, et al., J. Virol. 67(10): 5911-21, 1993). Thus, for Ad2 and Ad5, this would be an upper packaging limit of about 38 kb of DNA.

Adenovirus DNA also includes inverted terminal repeat sequences (ITRs) ranging in size from about 100 to 150 bp, depending on the serotype. The inverted repeats permit single strands of viral DNA to circularize by base-pairing of their terminal sequences to form base-paired “panhandle” structures that are required for replication of the viral DNA. For efficient packaging, the ITRs and the packaging signal (a few hundred bp in length) comprise the “minimum requirement” for replication and packaging of a genomic nucleic acid into an adenovirus particle. Helper-dependent vectors lacking all viral ORFs but including these essential cis elements (the ITRs and contiguous packaging sequence) have been constructed,

Ad vectors have several distinct advantages as gene delivery vehicles. For example, recombination of such vectors is rare; there are no known associations of human malignancies with adenoviral infections despite common human infection with adenoviruses; the genome may be manipulated to accommodate foreign genes of a fairly substantial size; and host proliferation is not required for expression of adenoviral proteins. Adenovirus (Ad)-based gene delivery vectors efficiently infect may different cells and tissues. This broad tropism, however, means that gene delivery cannot be directed to a specific target cell. A large fraction of intravenously administered adenovirus is retained by the liver, which could lead to undesirable side-effects. Adenovirus may potentiate immune responses. For example, Adenovirus type 5 (Ad5) also transduces dendritic cells, which present antigens very efficiently, thereby possibly exacerbating the immune response against the vector. It has been proposed that vectors with different targeting efficiencies might eliminate these problems, permitting a lower total particle dose and more specific targeting (see, e.g., U.S. application Ser. No. 09/482,682).

The wealth of information on adenovirus structure and mechanism of infection, its efficient infection of nondividing cells, and its large genetic capacity make adenovirus a popular gene therapy vector. The wide expression of receptors to which adenovirus binds makes targeting adenovirus vectors difficult. In particular, because of the widespread distribution of the Ad receptor (CAR), current adenoviral (Ad) vectors cannot be targeted to specific cell types (see, e.g., Bergelson et al. (1997) Science 275:1320-1323; Tomko et al. (1997) Proc.Natl.Acad.Sci. USA 94:3352-3356). Moreover, CAR and/or internalization receptors (av integrins) (see, Wickham et al. (1993) Cell 73:309-319) are absent or present at low levels on some cell types, rendering them resistant to Ad-mediated gene delivery.

Approximately 20% of all gene therapy clinical trials, registered with the NIH Recombinant DNA Advisory Committee, use replication-deficient adenovirus vectors (Office of Recombinant DNA Database). While successes have been reported, especially in the area of tumor management (see, e.g., Bilbao et al. (1998) Adv.Exp.Med.Biol. 451:365-374; Gomez-Navarro et al. (1999) Eur.J.Cancer 35:867-885), the use of adenovirus gene delivery vectors has been hampered by host inflammatory responses to the virus or encoded transgene products (Kay et al. (1997) Proc.Natl.Acad.Sci. USA 94:12744-12746). In addition, some cell types lack either the Ad attachment receptor, Coxsackie Adenovirus Receptor (CAR; Bergelson et al. (1997) Science 275:1320-1323; Tomko et al. (1997) Proc.Natl.Acad.Sci. USA 94:3352-3356) or integrins αvβ3 and αvβ5, which serve as virus internalization receptors (Wickham et al. (1993) Cell 73:309-319). For example, because airway epithelia do not express CAR and integrins on their apical surface (Goldman et al. (1995) J. Virol. 69:5951-5958; Grubb et al. (1994) Nature 371:802-806), clinical trials for the treatment of cystic fibrosis reported variable, generally low, efficacy (Zabner et al (1993) Cell 75:207-216; Crystal et al. (1994) Nature genet. 4:42-51) using adenoviral vectors.

Thus, while adenoviral vectors and others, hold much promise for therapeutic applications, their usefulness is limited by the widespread tissue distribution of CAR, which restricts delivery to specific cell types, and also by the absence of CAR and/or αv integrin receptors on certain cells in vivo. There have been attempts to overcome these limitations by modifying one or more of the Ad outer capsid proteins in order to retarget vectors to different cell receptor. While improvements in gene delivery have been realized, each method has its limitation. Also, the underlying factors that promote gene delivery have not been clearly defined, which has impeded further progress in Ad vector development; and the specificity of targeting requires further improvement.

Hence, there is a need to improve delivery and targeting of gene delivery vectors, including adenoviral gene delivery vectors, and to understand the underlying basis therefor. Therefore, it is an object herein to provide methods and vectors that can specifically target specific cells and tissues and that provide improved delivery and internalization of such vectors. It is also an object herein to identify the underlying mechanism for internalization and to provide delivery methods and delivery vectors based thereon.

### SUMMARY OF THE INVENTION

A vector targeting method that takes advantage of the common cell signaling pathways initiated by ligation of αv integrins and cell surface proteins and receptors that upon ligand interaction activate phosphatidylinositol 3 kinases (PI3K) or the phosphatidylinositol 3 (PI3) signalling pathway is provided. Bifunctional molecules (or multifunctional molecules) for effecting the targeting and complexes that contain the bifunctional molecules conjugated to a viral particle or bacterial surface protein are provided. Methods of gene delivery and gene therapy are also provided. In a preferred embodiment the virus for delivery and to which the bifunctional molecule specifically binds is adenovirus or adeno-associated virus. More preferably the virus is adenovirus, including fiberless viral particles.

In particular, bifunctional molecules that contain an antibody or antigen-binding portion thereof and a targeting agent are provided. The antibody specifically binds to an antigen in a protein that binds to αᵥ integrin; and the targeting agent specifically binds to a cell surface protein that activates the phosphatidylinositol 3 (PI3) signaling pathway. In particular, the targeting agent binds to a cell surface protein that triggers phosphatidylinositol-3-OH kinase (PI3K) activation.

Thus, the bifunctional molecules include a targeting agent, which is a moiety that specifically binds to a cell surface protein that triggers activation of PI3K, and a binding portion (designated “P” herein) that specifically binds to a protein on a viral particle or bacterial cell surface. Generally such viral or bacterial surface protein specifically binds to an αv integrin or other protein on the surface of a targeted cell that facilitates or effects internalization of the virus or bacterial into the cell.

The bifunctional molecules optionally include a linker or plurality of linkers that links the antibody or antigen-binding portion thereof to the targeting agent. The linker can be a peptide, preferably of from 2 to 100, more preferably 3 to 50, more preferably 5 to 20 amino acids, a single amino acid, or a chemical linker, such as those produced by reaction with crosslinking agents or heterobifunctional crosslinking reagents. The bifunctional molecules can be fusion proteins, chemical conjugates or mixtures thereof, a single amino acid or a peptide.

Thus the bifunctional agents can be represented by the formula:

(P)ₙ—(L)_{q}—(TA)ₘ, where m and n are integers of 1 or higher, and are generally 1, and q is 0 or an integer of one or more, and is generally 1 or 2. In instances where either or both of n and m are greater than 1, the resulting molecule is technically a multifunctional molecule. Each P and each TA do can be the same or different.

The protein that binds to αᵥ integrin is a viral protein or a bacterial protein that interacts with αᵥ integrins for internalization of the respective virus or bacteria. It is with such protein that the “P” moiety of the bifunctional molecule interacts. The P moiety is generally an antibody or portion thereof or recombinant molecule having the binding specificity of an antibody. The antibody or antigen-binding portion of the bifunctional molecule specifically binds to such protein, which for example is a viral protein, such as the penton protein of adenovirus.

The antibody or antigen-binding portion of the bifunctional molecule can include a heavy chain or a portion thereof sufficient for antigen-binding fused to the targeting agent; or is an Fab or Fab′2 fragment, or is a synthetic or recombinant molecule that contains antibody fragments, such as portions of the heavy chain and light chain variable regions sufficient for specific interaction with the antigen.

The targeted cell surface protein is any cell surface protein in which binding thereto facilitates internalization, particularly via the signaling pathway that involves PI3K. Such surface proteins, include, but are not limited to cytokine receptors, growth hormone receptors and other non-steroidal hormone receptors, such as a PDGF receptor, an IGF-1 receptor, an EGF receptor, a member of the FGF receptor family, a TNF receptor, a CSF-1 receptor, an insulin receptor, an IGF-1 receptor, an NGF receptor, an II-2 receptor, an II-3 receptor, an II-4 receptor, an IgM receptor, a CD4 receptor, a CD2 receptor, a CD3/T cell receptor, a G protein linked thrombin receptor, an ATP receptor, an fMLP receptor, and tyrosine kinase receptors that, when activated, result in increased accumulation of Ptdins(3, 4, 5)P3, receptors associated with the src family non-receptor tyrosine kinases that stimulate PI3Ks to lead to phosphatidyl-inositol(3, 4, 5)P3 (Ptdins(3, 4, 5)P3) accumulation.

In exemplified embodiments, growth factor/cytokines, such as TNF-α, IGF-1, SCF, PDGF and EGF, hormones, such as insulin, and other molecules or portions thereof that trigger phosphatidylinositol-3-OH kinase (PI3K) activation, a signaling molecule involved in adenovirus internalization, are fused to a monoclonal antibody specific for the viral particle protein that interacts with αv integrins, which in the case of adenovirus the viral penton base. Ad vectors complexed with these bifunctional mAbs exhibit increased gene delivery of about 10-50 fold to human melanoma cells lacking αv integrins. The bifunctional Mabs also enhance gene delivery by fiberless adenovirus particles, which cannot bind to CAR. Improved gene delivery correlates with increased virus internalization and attachment as well as PI3K activity. The use of bifunctional mAbs to trigger specific cell signaling pathways offers a widely applicable method for bypassing the normal Ad receptors in gene delivery and potentially increasing the selectivity of gene transfer.

Isolated nucleic acid molecule(s) that encode the bifunctional molecules are also provided. In some instances, in which the “P” moiety is an antibody or portion thereof, the bifunctional molecule includes two chains, which can be separately expressed and the reconstituted, such as by the selected expression system. Preferred expression systems are baculovirus systems.

Also provided are combinations that include a bifunctional molecule and a viral or bacterial vector. The components of the combinations may be separate or combined in a single composition in which the bifunctional molecules have been complexed with the viral particles or bacterial cells. Kits, containing the combinations optionally include instructions for administration and/or complexing, are also provided. Upon complexing of a bifunctional molecule and a viral or bacterial vector, the resulting complex can be used for delivery of gene products, such as for therapeutics, gene therapy and or for production of transgenic animals. Preferred delivery vectors herein are adenovirus vectors, including fiberless adenovirus vectors.

Methods of gene therapy by administration of the targeted gene delivery vectors that include the bifunctional molecules complexed with a viral particle or bacterial cells are also provided.

In exemplified embodiments, bifunctional molecules and complexes thereof with adenovirus delivery vectors are provided. In an exemplary embodiment, a bifunctional molecule that recognizes the RGD motif in the penton base protein of adenovirus is fused to the mature form of a receptor targeting molecule, such as TNF-α. The bifunctional molecules were expressed in insect cells using a non-lytic baculovirus expression system. In particular, the bifunctional molecule was produced from a monoclonal antibody, designated DAV-1, that specifically interacts with penton base on the surface of adenovirus strains, including Ad2, Ad3, Ad4 and Ad5. In particular, this antibody includes sequence of amino acids set forth in SEQ ID No. 2 or SEQ ID No. 6 or a sufficient portion thereof for antigen recognition or is encoded by nucleic acid that hybridizes along its full length under conditions of low stringency, more preferably moderate stringency, most preferably high stringency to a sufficient portion of SEQ ID Nos. 1 or 5 to encode an antigen-binding portion of the antibody. The antibody or portion thereof also include light chain that includes all or a portion of the sequence of amino acids set forth in SEQ ID No. 4 or nucleic acid that hybridizes along its full length under conditions of low stringency, more preferably moderate stringency, most preferably high stringency to a sufficient portion of the SEQ ID No. 3 so that the resulting molecule encodes an antigen-binding portion of the antibody.

The targeting agent or portion thereof can be selected from among, for example, is a tumor necrosis factor (TNF), a fibroblast growth factor (FGF), an insulin-like growth factor (IGF), a colony stimulating factor (CSF), insulin and a stem cell factor (SCF).

In exemplfied embodiments, bifunctional molecules were capable of enhancing infection of M21-L12 melanoma cells, which lack αv integrins and are also resistant to TNFα killing. M21 cells are relatively resistant to transduction with adenovirus vectors. Incubation of Ad encoding lacZ with the original monoclonal antibody alone had little effect enhancing gene delivery. In contrast, preincubation of Ad.lacZ particles with the bifunctional antibody produced a 20-fold increase in virus-mediated gene delivery. Enhanced virus infection by the bifunctional antibodies was due to a combination of increased virus binding and internalization. Enhanced internalization resulted from increased activation of PI3K through TNFα receptor ligation. Other bifunctional molecules containing receptor ligands were capable of activating PI3K and enhancing gene delivery.

These results demonstrate that activation of receptors that activate PI3K bypasses the requirement for αv integrins or, for adenovirus, CAR to promote virus entry. Receptor bypass was highly effective when cytokine or growth factors were activated in close proximity to bound virus particles.

### DETAILED DESCRIPTION OF THE INVENTION

A. Definitions

Unless defined otherwise, all technical and scientific terms used herein have the same meaning as is commonly understood by one of skill in the art to which this invention belongs. All patents, applications, published applications and other publications and sequences from GenBank and other data bases referred to anywhere in the disclosure herein are incorporated by reference in their entirety.

As used herein, the amino acids, which occur in the various amino acid sequences appearing herein, are identified according to their well-known, three-letter or one-letter abbreviations. The nucleotides, which occur in the various DNA fragments, are designated with the standard single-letter designations used routinely in the art (see, Table 1).

As used herein, amino acid residue refers to an amino acid formed upon chemical digestion (hydrolysis) of a polypeptide at its peptide linkages. The amino acid residues described herein are preferably in the “L” isomeric form. However, residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. §§1.821-1.822, abbreviations for amino acid residues are shown in the following Table:

TABLE 1
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left to right orientation in the conventional direction of amino-terminus to carboxyl-terminus. In addition, the phrase “amino acid residue” is broadly defined to include the amino acids listed in the Table of Correspondence and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, em., Watson et al. Molecular Biology of the Gene, 4th Edition, 1987, The Bejacmin/Cummings Pub. co., p.224).

Such substitutions are preferably made in accordance with those set forth in TABLE 2 as follows:

TABLE 2

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions are also permissible and may be determined empirically or in accord with known conservative substitutions.

As used herein, a complementing plasmid describes plasmid vectors that deliver nucleic acids into a packaging cell line for stable integration into a chromosome in the cellular genome.

As used herein, a delivery plasmid is a plasmid vector that carries or delivers nucleic acids encoding a therapeutic gene or gene that encodes a therapeutic product or a precursor thereof or a regulatory gene or other factor that results in a therapeutic effect when delivered in vivo in or into a cell line, such as, but not limited to a packaging cell line, to propagate therapeutic viral vectors.

As used herein, a variety of vectors are described. For example, one vector is used to deliver particular nucleic acid molecules into a packaging cell line for stable integration into a chromosome. These types of vectors are generally identified herein as complementing plasmids. A further type of vector described herein carries or delivers nucleic acid molecules in or into a cell line (e.g., a packaging cell line) for the purpose of propagating therapeutic viral vectors; hence, these vectors are generally referred to herein as delivery plasmids. A third “type” of vector described herein is used to carry nucleic acid molecules encoding therapeutic proteins or polypeptides or regulatory proteins or are regulatory sequences to specific cells or cell types in a subject in need of treatment; these vectors are generally identified herein as therapeutic viral vectors or recombinant adenoviral vectors or viral Ad-derived vectors and are in the form of a virus particle encapsulating a viral nucleic acid containing an expression cassette for expressing the therapeutic gene.

As used herein, a DNA or nucleic acid homolog refers to a nucleic acid that includes a preselected conserved nucleotide sequence, such as a sequence encoding a therapeutic polypeptide. By the term “substantially homologous” is meant having at least 80%, preferably at least 90%, most preferably at least 95% homology therewith or a less percentage of homology or identity and conserved biological activity or function.

The terms “homology” and “identity” are often used interchangeably. In this regard, percent homology or identity may be determined, for example, by comparing sequence information using a GAP computer program. The GAP program utilizes the alignment method of Needleman and Wunsch (J. Mol. Biol. 48:443 (1970), as revised by Smith and Waterman (Adv. Appl. Math. 2:482 (1981). Briefly, the GAP program defines similarity as the number of aligned symbols (i.e., nucleotides or amino acids) which are similar, divided by the total number of symbols in the shorter of the two sequences. The preferred default parameters for the GAP program may include: (1) a unary comparison matrix (containing a value of 1 for identities and 0 for non-identities) and the weighted comparison matrix of Gribskov and Burgess, Nucl. Acids Res. 14:6745 (1986), as described by Schwartz and Dayhoff, eds., ATLAS OF PROTEIN SEQUENCEAND STRUCTURE, National Biomedical Research Foundation, pp. 353-358 (1979); (2) a penalty of 3.0 for each gap and an additional 0.10 penalty for each symbol in each gap; and (3) no penalty for end gaps.

Whether any two nucleic acid molecules have nucleotide sequences that are at least 80%, 85%, 90%, 95%, 96%, 97%, 98% or 99% “identical” can be determined using known computer algorithms such as the “FAST A” program, using for example, the default parameters as in Pearson and Lipman, Proc. Natl. Acad. Sci. USA 85:2444 (1988). Alternatively the BLAST function of the National Center for Biotechnology Information database may be used to determine identity

In general, sequences are aligned so that the highest order match is obtained. “Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H. & Lipton, D., SIAM J Applied Math 48:1073 (1988)). Methods commonly employed to determine identity or similarity between two sequences include, but are not limited to, those disclosed in Guide to Huge Computers, Martin J. Bishop, ed., Academic Press, San Diego, 1994, and Carillo, H. & Lipton, D., SIAM J Applied Math 48:1073 (1988). Methods to determine identity and similarity are codified in computer programs. Preferred computer program methods to determine identity and similarity between two sequences include, but are not limited to, GCG program package (Devereux, J., et al., Nucleic Acids Research 12(I):387 (1984)), BLASTP, BLASTN, FASTA (Atschul, S. F., et al., J Molec Biol 215:403 (1990)).

Therefore, as used herein, the term “identity” represents a comparison between a test and a reference polypeptide or polynucleotide. For example, a test polypeptide may be defined as any polypeptide that is 90% or more identical to a reference polypeptide.

As used herein, the term at least “90% identical to” refers to percent identities from 90 to 99.99 relative to the reference polypeptides. Identity at a level of 90% or more is indicative of the fact that, assuming for exemplification purposes a test and reference polynucleotide length of 100 amino acids are compared. No more than 10% (i.e., 10 out of 100) amino acids in the test polypeptide differs from that of the reference polypeptides. Similar comparisons may be made between a test and reference polynucleotides. Such differences may be represented as point mutations randomly distributed over the entire length of an amino acid sequence or they may be clustered in one or more locations of varying length up to the maximum allowable, e.g. 10/100 amino acid difference (approximately 90% identity). Differences are defined as nucleic acid or amino acid substitutions, or deletions.

As used herein, stringency conditions refer to the washing conditions for removing the non-specific probes and conditions that are equivalent to either high, medium, or low stringency as described below:

1) high stringency: 0.1×SSPE, 0.1% SDS, 65° C.

2) medium stringency: 0.2×SSPE, 0.1% SDS, 50° C.

3) low stringency: 1.0×SSPE, 0.1% SDS, 50° C.

It is understood that equivalent stringencies may be achieved using alternative buffers, salts and temperatures.

As used herein, genetic therapy involves the transfer of heterologous DNA to the certain cells, target cells, of a mammal, particularly a human, with a disorder or conditions for which such therapy is sought. The DNA is introduced into the selected target cells in a manner such that the heterologous DNA is expressed and a therapeutic product encoded thereby is produced. Alternatively, the heterologous DNA may in some manner mediate expression of DNA that encodes the therapeutic product, it may encode a product, such as a peptide or RNA that in some manner mediates, directly or indirectly, expression of a therapeutic product. Genetic therapy may also be used to nucleic acid encoding a gene product replace a defective gene or supplement a gene product produced by the mammal or the cell in which it is introduced. The introduced nucleic acid may encode a therapeutic compound, such as a growth factor inhibitor thereof, or a tumor necrosis factor or inhibitor thereof, such as a receptor therefor, that is not normally produced in the mammalian host or that is not produced in therapeutically effective amounts or at a therapeutically useful time. The heterologous DNA encoding the therapeutic product may be modified prior to introduction into the cells of the afflicted host in order to enhance or otherwise alter the product or expression thereof.

As used herein, heterologous DNA is DNA that encodes RNA and proteins that are not normally produced in vivo by the cell in which it is expressed or that mediates or encodes mediators that alter expression of endogenous DNA by affecting transcription, translation, or other regulatable biochemical processes. Heterologous DNA may also be referred to as foreign DNA. Any DNA that one of skill in the art would recognize or consider as heterologous or foreign to the cell in which is expressed is herein encompassed by heterologous DNA. Examples of heterologous DNA include, but are not limited to, DNA that encodes traceable marker proteins, such as a protein that confers drug resistance, DNA that encodes therapeutically effective substances, such as anti-cancer agents, enzymes and hormones, and DNA that encodes other types of proteins, such as antibodies. Antibodies that are encoded by heterologous DNA may be secreted or expressed on the surface of the cell in which the heterologous DNA has been introduced.

Hence, herein heterologous DNA or foreign DNA, refers to a DNA molecule not present in the exact orientation and position as the counterpart DNA molecule found in the corresponding wild-type adenovirus. It may also refer to a DNA molecule from another organism or species (i.e., exogenous) or from another Ad serotype.

As used herein, a therapeutically effective product is a product that is encoded by heterologous DNA that, upon introduction of the DNA into a host, a product is expressed that effectively ameliorates or eliminates the symptoms, manifestations of an inherited or acquired disease or that cures said disease.

Typically, DNA encoding the desired heterologous DNA is cloned into a plasmid vector and introduced by routine methods, such as calcium-phosphate mediated DNA uptake (see, (1981) Somat. Cell. Mol. Genet. 7:603-616) or microinjection, into producer cells, such as packaging cells. After amplification in producer cells, the vectors that contain the heterologous DNA are introduced into selected target cells.

As used herein, an expression or delivery vector refers to any plasmid or virus into which a foreign or heterologous DNA may be inserted for expression in a suitable host cell—i.e., the protein or polypeptide encoded by the DNA is synthesized in the host cell's system. Vectors capable of directing the expression of DNA segments (genes) encoding one or more proteins are referred to herein as “expression vectors.” Also included are vectors that allow cloning of cDNA (complementary DNA) from mRNA produced using reverse transcriptase.

As used herein, a gene is a nucleic acid molecule whose nucleotide sequence encodes RNA or polypeptide. A gene can be either RNA or DNA. Genes may include regions preceding and following the coding region (leader and trailer) as well as intervening sequences (introns) between individual coding segments (exons).

As used herein, tropism with reference to a adenovirus refers to the selective infectivity or binding that is conferred on the particle by the fiber protein, particularly the C-terminus portion that comprises the knob.

As used herein, isolated with reference to a nucleic acid molecule or polypeptide or other biomolecule means that the nucleic acid or polypeptide has separated from the genetic environment from which the polypeptide or nucleic acid were obtained. It may also mean altered from the natural state. For example, a polynucleotide or a polypeptide naturally present in a living animal is not “isolated,” but the same polynucleotide or polypeptide separated from the coexisting materials of its natural state is “isolated”, as the term is employed herein. Thus, a polypeptide or polynucleotide produced and/or contained within a recombinant host cell is considered isolated. Also intended as an “isolated polypeptide” or an “isolated polynucleotide” are polypeptides or polynucleotides that have been purified, partially or substantially, from a recombinant host cell or from a native source. For example, a recombinantly produced version of a compounds can be substantially purified by the one-step method described in Smith and Johnson, Gene 67:31-40 (1988). The terms isolated and purified are sometimes used interchangeably.

Thus, by “isolated” is meant that the nucleic is free of the coding sequences of those genes that, in the naturally-occurring genome of the organism (if any) immediately flank the gene encoding the nucleic acid of interest. Isolated DNA may be single-stranded or double-stranded, and may be genomic DNA, cDNA, recombinant hybrid DNA, or synthetic DNA. It may be identical to a native DNA sequence, or may differ from such sequence by the deletion, addition, or substitution of one or more nucleotides.

Isolated or purified as it refers to preparations made from biological cells or hosts means any cell extract containing the indicated DNA or protein including a crude extract of the DNA or protein of interest. For example, in the case of a protein, a purified preparation can be obtained following an individual technique or a series of preparative or biochemical techniques and the DNA or protein of interest can be present at various degrees of purity in these preparations. The procedures may include for example, but are not limited to, ammonium sulfate fractionation, gel filtration, ion exchange change chromatography, affinity chromatography, density gradient centrifugation and electrophoresis.

A preparation of DNA or protein that is “substantially pure” or “isolated” should be understood to mean a preparation free from naturally occurring materials with which such DNA or protein is normally associated in nature. “Essentially pure” should be understood to mean a “highly” purified preparation that contains at least 95% of the DNA or protein of interest.

A cell extract that contains the DNA or protein of interest should be understood to mean a homogenate preparation or cell-free preparation obtained from cells that express the protein or contain the DNA of interest. The term “cell extract” is intended to include culture media, especially spent culture media from which the cells have been removed.

As used herein, a packaging cell line is a cell line that provides a missing gene product or its equivalent.

As used herein, an adenovirus viral particle is the minimal structural or functional unit of a virus. A virus can refer to a single particle, a stock of particles or a viral genome. The adenovirus (Ad) particle is relatively complex and may be resolved into various substructures.

As used herein, “penton” or “penton complex” are preferentially used herein to designate a complex of penton base and fiber. The term “penton” may also be used to indicate penton base, as well as penton complex. The meaning of the term “penton” alone should be clear from the context within which it is used.

As used herein, a plasmid refers to an autonomous self-replicating extrachromosomal circular nucleic acid molecule, typically DNA.

As used herein, a post-transcription regulatory element (PRE) is a regulatory element found in viral or cellular messenger RNA that is not spliced, i.e. intronless messages. Examples include, but are not limited to, human hepatitis virus, woodchuck hepatitis virus, the TK gene and mouse histone gene. The PRE may be placed before a polyA sequence and after a heterologous DNA sequence.

As used herein, pseudotyping describes the production of adenoviral vectors having modified capsid protein or capsid proteins from a different serotype than the serotype of the vector itself. One example, is the production of an adenovirus 5 vector particle containing an Ad37 fiber protein. This may be accomplished by producing the adenoviral vector in packaging cell lines expressing different fiber proteins.

As used herein, promoters of interest herein may be inducible or constitutive. Inducible promoters will initiate transcription only in the presence of an additional molecule; constitutive promoters do not require the presence of any additional molecule to regulate gene expression. a regulatable or inducible promoter may also be described as a promoter where the rate or extent of RNA polymerase binding and initiation is modulated by external stimuli. Such stimuli include, but are not limited to various compounds or compositions, light, heat, stress and chemical energy sources. Inducible, suppressible and repressible promoters are considered regulatable promoters. Preferred promoters herein, are promoters that are selectively expressed in ocular cells, particularly photoreceptor cells.

As used herein, receptor refers to a biologically active molecule that specifically binds to (or with) other molecules. The term “receptor protein” may be used to more specifically indicate the proteinaceous nature of a specific receptor.

As used herein, recombinant refers to any progeny formed as the result of genetic engineering. This may also be used to describe a virus formed by recombination of plasmids in a packaging cell.

As used herein, a transgene or therapeutic nucleic acid molecule includes DNA and RNA molecules encoding an RNA or polypeptide. Such molecules may be “native” or naturally-derived sequences; they may also be “non-native” or “foreign” that are naturally- or recombinantly-derived. The term “transgene,” which may be used interchangeably herein with the term “therapeutic nucleic acid molecule,” is often used to describe a heterologous or foreign (exogenous) gene that is carried by a viral vector and transduced into a host cell.

Therefore, therapeutic nucleotide nucleic acid molecules include antisense sequences or nucleotide sequences which may be transcribed into antisense sequences. Therapeutic nucleotide sequences (or transgenes) all include nucleic acid molecules that function to produce a desired effect in the cell or cell nucleus into which said therapeutic sequences are delivered. For example, a therapeutic nucleic acid molecule can include a sequence of nucleotides that encodes a functional protein intended for delivery into a cell which is unable to produce that functional protein.

As used herein, a promoter region refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences may be cis acting or may be responsive to trans acting factors. Promoters, depending upon the nature of the regulation, may be constitutive or regulated.

As used herein, the phrase “operatively linked” generally means the sequences or segments have been covalently joined into one piece of DNA, whether in single or double stranded form, whereby control sequences on one segment control expression or replication or other such control of other segments. The two segments are not necessarily contiguous.

As used herein, exogenous encompasses any therapeutic composition that is administered by the therapeutic methods provided herein. Thus, exogenous may also be referred to herein as foreign, or non-native or other equivalent expression.

As used herein, antibody refers to an immunoglobulin, whether natural or partially or wholly synthetically produced, including any derivative thereof that retains the specific binding ability the antibody. Hence antibody includes any protein having a binding domain that is homologous or substantially homologous to an immunoglobulin binding domain. Antibodies include members of any immunoglobulin claims, including IgG, IgM, IgA, IgD and IgE.

As used herein, antibody fragment refers to any derivative of an antibody that is less then full length, retaining at least a portion of the full-length antibody's specific binding ability. Examples of antibody fragments include,but are not limited to, Fab, Fab′, F(ab)₂, single-chain Fvs (scFV), FV, dsFV diabody and Fd fragments and other recombinant form of antibodies that retain or exhibit binding specificity.

The fragment can include multiple chains linked together, such as by disulfide bridges. An antibody fragment generally contains at least about 50 amino acids and typically at least 200 amino acids. For purposes herein, any fragment that retains specific binding for a αv integrin binding protein is contemplated. Such fragments may be produced by chemical or recombinant means.

As used herein, an Fv antibody fragment is composed of one variable heavy domain (V_{H}) and one variable light domain linked by noncovalent interactions.

As used herein, a dsFV refers to an Fv with an engineered intermolecular disulfide bond, which stabilizes the V_{H}-V_{L} pair.

As used herein, an F(ab)₂ fragment is an antibody fragment that results from digestion of an immunoglobulin with pepsin at pH 4.0-4.5; it may be recombinantly produced.

Thus, with reference to the DAV-1 antibody exemplified herein, the Fab portion is the entire light chain and amino acids 1-229 of the DAV-1 heavy chain (SEQ ID Nos. 1 and 2). The Fab fragment is involved in antigen binding, with the exception of the first 19 amino acids which constitute the signal peptide sequence for secretion of the antibody. Fab fragments can be generated by digestion with papain.

The Fab′2 portion is the Fab fragment and the hinge region, which connects the Fab antigen-binding portion with the Fc portion which is involved in complement activation and macrophage binding. Amino acids 230-242 of the DAV-1 heavy fragment constitute the hinge region of DAV-1. The Fab′2 fragments used in this manuscript that were generated by cloning comprise amino acids 1-247 of the DAV-1 heavy chain sequence. Fab′2 fragments can be generated by digestion with pepsin.

As used herein, Fab fragments is an antibody fragment that results from digestion of an immunoglobulin with papain; it may be recombinantly produced.

As used herein, scFVs refer to antibody fragments that contain a variable light chain (V_{L}) and variable heavy chain (V_{H}) covalently connected by a polypeptide linker in any order. The linker is of a length such that the two variable domains are bridged without substantial interference. Preferred linkers are (Gly-Ser)ₙ residues with some Glu or Lys residues dispersed throughout to increase solubility.

As used herein, diabodies are dimeric scFV; diabodies typically have shorter peptide linkers than scFvs, and they preferentially dimerize.

As used herein, humanized antibodies refer to antibodies that are modified to include “human” sequences of amino acids so that administration to a human will not provoke an immune response. Methods for preparation of such antibodies are known. For example, the hybridoma that expresses the monoclonal antibody is altered by recombinant DNA techniques to express an antibody in which the amino acid composition of the non-variable regions is based on human antibodies. Computer programs have been designed to identify such regions. For human therapy, humanized antibodies are preferred.

As used herein, production by recombinant means by using recombinant DNA methods means the use of the well known methods of molecular biology for expressing proteins encoded by cloned DNA, including cloning expression of genes and methods, such as gene shuffling and phage display with screening for desired specificities.

As used herein, the term “conjugated” refers stable attachment, such ionic or covalent attachment.

As used herein, a composition refers to any mixture of two or more products or compounds. It may be a solution, a suspension, liquid, powder, a paste, aqueous, non-aqueous or any combination thereof.

As used herein, a combination refers to any association between two or more items.

As used herein, fluid refers to any composition that can flow. Fluids thus encompass compositions that are in the form of semi-solids, pastes, solutions, aqueous mixtures, gels, lotions, creams and other such compositions.

As used herein, substantially identical to a product means sufficiently similar so that the property of interest is sufficiently unchanged so that the substantially identical product can be used in place of the product.

B. Adenovirus Delivery and Internalization

Attachment and Internalization

Adenovirus attachment to cells is mediated by the elongated fiber, which mediates initial attachment of the virus to cell receptors. The fiber is a homotrimeric protein with an elongated central shaft domain that varies in length among different virus serotypes. The C terminus of the protein contains a globular domain known as the knob, which is responsible for receptor interaction. The N terminus anchors the protein to the virus surface via interaction with the penton base protein.

The cellular receptor recognized by fiber is known as Coxsackie Adenovirus Receptor (CAR), which is a member of the Ig superfamily and contains two extracellular domains. The N-terminal domain is sufficient to mediate fiber interaction. Secondary interactions of the virus penton base protein with αv integrins promote virus internalization into clathrin-coated pits and endosomes.

Virions are capable of disrupting the early endosome allowing the majority of the virion particle to escape into the cytoplasm. Viral particles are then transported along microtubules to the nuclear pore complex and virion DNA is translocated into the nucleus.

Ad enter cells by a clathrin-coated pit pathway. Ad internalization is complex process that is found herein to be similar in some respects to that described for cell invasion by certain pathogenic bacteria. Clustering of αv integrins (not CAR) results in the formation of a cytoplasmic signaling complex involving at least three major components (cSRC, CAS, PI3K). This signaling complex is capable of further activation of the Rho family of small GTPases (RAC, CDC42) whose activation ultimately results in the reorganization of actin cytoskeleton and enhanced virus internalization.

Adenovirus internalization via the clathrin-coated pit pathway (Patterson et al. (1983) J. Gen. Virol. 64:1091-1099; Wang et al. (1998) J. Virol. 72:3455-3458 ) requires activation of several signaling molecules including phospatidylinositol 3-OH kinase (PI3K; Li et al. (1998) J. Virol. 72:2055-2061) and the Rho family of small GTPases (Li et al. (1998) J. Virol. 72:8806-8812). The major downstream target of this signaling pathway is the actin cytoskeleton, which promotes virus uptake.

PI3Ks

The phosphatidylinositol 3-kinases (PI3 kinases or PI3Ks) represent a ubiquitous family of heterodimeric lipid kinases that are found in association with the cytoplasmic domain of hormone and growth factor receptors and oncogene products. Phosphoinositide 3-OH-kinases (PI3Ks) constitute a large family of enzymes capable of 3-phosphorylating at least one of the cellular phosphoinositides (Whitman et al. (1988) Nature 332:644-646). 3-phosphorylated phosphoinositides are found in all higher eukaryotic cells.

PI3Ks act as downstream effectors of the above-noted receptors, are recruited upon receptor stimulation and mediate the activation of second messenger signaling pathways through the production of phosphorylated derivatives of inositol. PI3Ks have been implicated in many cellular activities including growth factor mediated cell transformation, mitogenesis, protein trafficking, cell survival and proliferation, DNA synthesis, apoptosis, neurite outgrowth, nitric oxide signaling and insulin-stimulated glucose transport.

PI3Ks phosphorylate phosphatidylinositol (Ptdlns) at the 3′-hydroxyl of the inositol ring and substrates include Ptdins, Ptlns(4)phosphate Ptdlns(4,5)bisphosphate and Ptdins(3,4,5)triphosphate; the major product is Ptdins(3,4,5)triphosphate (see, e.g., Fry (1994) Biochim. Biophys. Acta. 1226: 237-268). A PI3K and a lipid product of this enzyme, phosphatidylinositol(3,4,5)-triphosphate (hereinafter “Ptdins(3,4,5)P3”), are part of an important second messenger system in cellular signal transduction. Ptdlns(3,4,5) P3 appears to be a second messenger in extremely diverse signalling pathways.

As described herein, PI3K is a member of a family of lipid kinases that include a p85 regulatory subunit and a p110 catalytic subunit (Toker et al, Nature, 387:673 (1997)). The p85 subunit of PI3K binds directly to phosphorylated FAK (Chen et al., J. Biol. Chem., 271:26329 (1996)). The products of PI3K activation, phosphatidylinositol-3,4-biphosphate and phosphatidylinositol-3,4,5-triphosphate are increased in the plasma membrane of activated but not quiescent cells and have been proposed to act as second messengers for a number of cell functions including cell motility, the Ras pathway, vesicle trafficking and secretion, apoptosis, the movement of organelle membranes, shape alteration through rearrangement of cytoskeletal actin, transformation, chemotaxis, cell cycle progression and intracellular protein trafficking (Carpenter et al., Curr. Opin. Cell Biol., 8:153 (1996); Chou et al., Cell 85:573 (1996); Wennström et al., Curr. Biol., 4:385 (1994); Toker et al., Nature, 387:673 (1997)). The phospholipid second messengers phosphatidylinositol-3,4-biphosphate and phosphatidylinositol-3,4,5-triphosphate mediate the cell functions and processes by activation of protein kinase B and the small GTP-binding proteins Ras, Rho, Rac and CDC42 (Hall, Science, 279:509 (1998); Karlund et al., Science 275:1927 (1997); Tapon et al., Curr. Opin. Cell Biol., 9:86 (1995)). Activation of Rac and CDC42 induces polymerization of monomeric actin, resulting in the formation of a dense network of actin filaments underlying the plasma membrane, and the actin-rich regions form a variety of membrane extensions known as lamellipodia (“membrane ruffling”) and filopodia (“hairlike structures”) (Luo et al., Nature 379:837 (1996)). Activation of Rho is associated with the formation of actin-associated stress fibers (Nobes et al., Cell, 81:53 (1995); Ridley et al., Cell, 70:389 (1992)). The polymerized actin filaments maintain the architecture of the surface protrusions. This actin assembly initiated by activation of PI3K plays an important role in a number of biological and/or pathogenic processes including directed cell migration (Zigmond, Curr. Biol., 8:66 (1996)), axonal guidance in neuronal cells (Luo et al., Genes Dev., 8:1787 (1994)), and cell invasion by a number of different bacteria (Chen et al., Science, 274:2115 (1996); Hordijk et al., Science, 278:1464 (1998)). Recent studies have indicated that actin polymerization also facilitates clathrin-mediated endocytosis (Benmerah et al., J. Cell Biol., 140:1055 (1998); Lamaze et al., J. Biol. Chem., 272:20332 (1997); Witke et al., EMBO J., 17:967 (1998)).

A number of stimulatory agonists activate the PI3K-mediated signalling pathway. Many of the effects of insulin on glucose and lipid metabolism are elicited through activation of PI3K (Shepherd et al., Biochem. J., 333(3):471 (1998)). IGF-1, PDGF and EGF stimulate cell proliferation in astroglial cells through increased PI3K activity (Pomerance et al., J. Neurosci. Res., 40:737 (1995)). Insulin or IGF-1 induced membrane ruffling is mediated via activation of PI3K (Kotani et al., EMBO J., 13(10):2313 (1994)). PI3K-associated p85 associates with EGFR and PDGFR upon stimulation with EGF or PDGF, respectively (Hu et al., Mol. Cell. Biol., 12:981 (1992)). Fibronectin, insulin and PDGF stimulation of ILK (integrin-linked kinase) are dependent on the activity of PI3K (Delcommenne et al., Proc. Natl. Acad. Sci. USA 95:11211 (1998)). Stimulation of certain cell types with TNF-α has been shown to activate PI3K (Guo et al., J. Biol. Chem., 271:615 (1996)). PI3K is also activated by internalins, which are bacterial surface proteins involved in bacterial invasion (Ireton et al., Science, 274:780 (1996)).

Adenovirus endocytosis also requires activation of PI3K (Li et al., J. Virol. 72:2055 (1998)). Adenovirus endocytosis is a multistep process beginning with its attachment to cells via the elongated fiber protein which the cell surface protein receptor known as CAR (Bergelson et al., Science, 275:1320 (1997)). Secondary interactions of the virus penton base protein via their RGD motifs with αv integrins on the cell surface promotes virus internalization, via a receptor-mediated endocytosis pathway, into clathrin-coated pits and endosomes. While fiber binding is not required for PI3K activation, the interaction of adenovirus penton base protein to with αv integrins stimulates PI3K (Li et al., J. Virol. 72:2055 (1998)). Stimulation of PI3K has been shown to be essential to viral entry; when cells were treated with the PI3K-specific inhibitors wortmannin and LY294002, adenovirus internalization was inhibited (Li et al., J. Virol. 72:2055 (1998)). The clustering of the αv integrins results in the formation of a cytoplasmic signaling complex involving at least three major components: cSRC, CAS and PI3K. This signaling complex is capable of further activation of the Rho family of small GTPases (Rac, CDC42) whose activation ultimately results in the reorganization of the actin cytoskeleton and enhanced virus internalization.

Thus, the signaling pathways activated by growth factors/cytokine receptors, including tumor necrosis factor a (TNF-α), insulin-like growth factor 1 (IGF-1) and epidermal growth factor (EGF) receptors (Hotamisligil et al. (1994) Proc.Natl.Acad.Sci.USA 24:4854-4858; Guo et al. (1996) J.Biol.Chem. 271:615-618; Pomerance et al. (1995 J.Neurosci. 40:737-746; Kotani, et al. (1994) EMBO J. 13:2313-2321; and Hu et al. (1992) Mol. Cell Biol. 12:981-990) initiate signaling events that converge at PI3K activation.

It is shown herein that advantage can be taken of the similarity in signaling processes elicited by cell surface receptors that activate PI3 kinases and the αv integrin mediated viral internalization, providing the basis for the methods and vectors provided herein, which bypass Ad integrin interaction to facilitate gene delivery. It is shown herein that molecules that bind to and activate receptors that participate in the PI3 signalling pathway can be used to target linked moieties, such as adenoviral particles, to such receptors, which include hormone and growth factor receptors, other G-protein coupled receptors and receptors for various oncogenes, and effect internalization.

1. Bifunctional Molecules

Provided herein are bifunctional molecules that are conjugates of an agent, such as an antibody or fragment thereof, that specifically binds to av integrin-binding protein (referred to as component “P” herein) and an agent (referred to as TA) that targets moieties linked to the protein that specifically binds to the αv integrin-binding protein to cells that express surface receptors to which the targeting agent specifically binds. TA and P are joined directly, typically by covalent linkage, or are linked via a linker L.

For purposes herein these conjugates are referred to as bifunctional molecules and include the following components: (TA)ₙ, Pₘ and L_{q}, wherein n and m are integers of 1 or more, typically 1 or 2, and q is 0 or an integer of 1 or more. These components are selected such that the resulting molecules will bind to or interact with a protein on a viral particle or bacteria to form a complex and target the resulting complex to a cell surface protein that activates PI3K and that is recognized by the targeting agent such that the resulting complex is internalized. Where n and m are other than one, the resulting molecules are multifunctional, in such instances more than one TA and/or P moiety can be used, each may be the same or different. The components TA, P and L may be conjugated by covalent linkages, ionic linkages or any other bond resulting in attachment.

It is understood that the P and the targeting agent (or linker and targeted agent) may be linked in any order and through any appropriate linkage, as long as the resulting conjugate binds to a receptor to which targeted binds and internalizes the complexed viral particles or bacterial cells in cells bearing the receptor.

Generally, P is an antibody or fragment thereof that specifically binds to αv integrin binding proteins, such as the penton protein of adenovirus. P is preferably a monoclonal antibody or fragment thereof or synthetic antibody or recombinant protein that includes a sufficient portion of the antibody chains to specifically bind to a selected antigen. In this instance, the antigen is one present in a protein on a viral particle or bacterial surface that mediates attachment of the viral particle of bacterium to αv integrins.

a. Antigen-binding Portion

In preferred embodiments, the moiety (P) that binds to proteins that interact with αv integrins, is a monoclonal antibody or antigen-binding fragment thereof. Proteins, such as the penton protein of adenovirus, that interact with αv integrins to facilitate internalization of viral particles and bacteria occur on viral particles and bacterial cells surfaces. Antibodies specific therefor can be made by standard methods, such as using hybridoma technology or by making recombinant antibodies or fragments thereof and screening, such as through phage display technology, for binding to the viral or bacterial surface protein. The antibody or fragment thereof or recombinant version thereof, is then modified to include a targeting agent, such as a growth factor or hormone or other protein that binds to receptors that activate the PI3K signaling pathway. The resulting bifunctional molecule specifically binds to a viral or bacterial surface protein, such as penton, and also will specifically bind to a selected targeted cell that expresses the targeted receptors.

b. Targeting Agents

The targeting agents are those that are ligands, including hormones, growth factors and cytokines, that specifically bind to and activate receptors that activate the PI3K signaling pathway. Hence the targeting agent is chosen to bind to receptors. Selecting targeted receptors can be those that are overexpressed in a particular disorder, such as an angiogenic disorder, including cancers, and inflammatory disorders. PI3K activity in cells of hematopoietic lineage, particularly neutrophils, monocytes, and other types of leukocytes, is involved in many of the non-memory immune responses associated with acute and chronic inflammation.

Receptors include, but are not limited to, tyrosine kinase receptors which, when activated, result in increased accumulation of Ptdlns(3, 4, 5)P3, such as the PDGF receptor, the EGF receptor, members of the FGF receptor family, the CSF-1 receptor, the insulin receptor, the IGF-1 receptor, the SCF (stem cell factor) receptor, a TNF receptor, such as a TNF-α receptor, and the NGF receptor; receptors associated with the src family non-receptor tyrosine kinases that stimulate Ptdlns(3, 4, 5)P3 accumulation, such as the II-2 receptor, II-3 receptor, mIgM receptor, the CD4 receptor, the CD2 receptor, and the CD3/T cell receptor. Other receptors, such as the cytokine II-4 receptor and the G protein linked thrombin receptor, ATP receptor, and the fMLP receptor, that stimulate the activity of a PI3K, resulting in subsequent Ptdlns(3, 4, 5)P3 accumulation are contemplated herein.

The targeting agents are selected to bind to such cell surface proteins, which must then facilitate internalization of the targeting agent and anything linked thereto.

The resulting conjugates provided can be used to delivery genes and products to cells that express such receptors. Hence, this provides a means to specifically target genes and products to a wide array of cells and in a wide variety of organisms, including plants as well as animals, for effecting genetic therapy and/or delivering products to cells involved in a wide array of disorders and conditions.

2. Preparation of Bifunctional Molecules

Methods for preparation of antigen-binding proteins, such as antibodies and antigen-binding fragments thereof that bind to proteins that bind to αv integrins are known to those of skill in the art. The bifunctional molecules can be prepared by recombinant and/or chemical methods. Bifunctional molecules can be fusion proteins that can be prepared using standard recombinant methods. Bifunctional molecules can be prepared using heterobifunctional reagents and linkers or other suitable chemical conjugation agents. Preparation of bifunctional molecules is exemplified herein, and the exemplified methods can be adapted for preparation of any desired bifunctional molecules.

Plasmids and Host Cells for Expression of Constructs Encoding Bifunctional Molecules

Nucleic acid encoding the selected “P” moiety, such as antibody, generally a heavy chain or portion thereof, is inserted into a suitable vector and expressed in a suitable prokaryotic or eukaryotic host. For antibody expression, the light chain can be inserted into another plasmid for expression. Numerous suitable hosts and vectors are known and available to those of skill in this art and may be purchased commercially or constructed according to published protocols using well known and available starting materials. Suitable eukaryotic host cells include insect cells, yeast cells, and animal cells. Insect cells and bacterial host cells are presently preferred. Suitable prokaryotic host cells include E. coli, strains of Bacillus and Streptomyces. For purposes herein, baculovirus expression systems are preferred.

The DNA construct is introduced into a plasmid suitable for expression in the selected host. The sequences of nucleotides in the plasmids that are regulatory regions, such as promoters and operators, are operationally associated with one another for transcription. The sequence of nucleotides encoding the αv integrin binding protein (designated P) can also include DNA encoding a secretion signal, whereby the resulting peptide is a precursor protein. Secretion signals suitable for use are widely available and are well known in the art. Prokaryotic and eukaryotic secretion signals functional in E. coli, may be employed. The presently preferred secretion signals include, but are not limited to, those encoded by the following E. coli genes: ompA, ompT, ompF, ompC, beta-lactamase, pelB and bacterial alkaline phosphatase, and the like (von Heijne (1985) J. Mol. Biol. 184:99-105). In addition, the bacterial pelB gene secretion signal (Lei et al. (1987) J. Bacteriol. 169:4379), the phoA secretion signal, and the cek2 secretion signal, functional in insect cells, may be employed. The most preferred secretion signal for bacterial expression is the E. coli ompA secretion signal. For eukaryotic expression systems, particularly insect cell systems, the signals from secreted proteins, such as insulin, growth hormone, mellitin, and mammalian alkaline phosphatase are of interest herein. Other prokaryotic and eukaryotic secretion signals known to those of skill in the art may also be employed (see, eg., von Heijne (1985) J. Mol. Biol. 184:99-105). Using the methods described herein, one of skill in the art can substitute secretion signals that are functional in either yeast, insect or mammalian cells to secrete the heterologous protein from those cells. The resulting processed protein may be recovered from the periplasmic space or the fermentation medium or growth medium.

The plasmids can include a selectable marker gene or genes that are functional in the host. A selectable marker gene includes any gene that confers a phenotype on bacteria that allows transformed bacterial cells to be identified and selectively grown from among a vast majority of untransformed cells. Suitable selectable marker genes for bacterial hosts, for example, include the ampicillin resistance gene (Amp^{r}), tetracycline resistance gene (Tc^{r}) and the kanamycin resistance gene (Kan^{r}). The kanamycin resistance gene is presently preferred.

The plasmids used herein preferably include a promoter in operable association with the DNA encoding the protein or polypeptide of interest and are designed for expression of proteins in a bacterial host. It has been found that tightly regulatable promoters are preferred for expression of saporin. Suitable promoters for expression of proteins and polypeptides herein are widely available and are well known in the art. For expression of the proteins such promoters are inserted in a plasmid in operative linkage with a control region such as the lac operon. Preferred promoter regions are those that are inducible and functional in E. coli or early genes in vectors of viral origin. Examples of suitable inducible promoters and promoter regions include, but are not limited to: the E. coli lac operator responsive to isopropyl β-D-thiogalactopyranoside (IPTG; see, et al. Nakamura et al. (1979) Cell 18:1109-1117); the metallothionein promoter metal-regulatory-elements responsive to heavy-metal (e.g., zinc) induction (see, e.g., U.S. Pat. No. 4,870,009 to Evans et al.); the phage T7lac promoter responsive to IPTG (see, em, U.S. Pat. No. 4,952,496; and Studier et al. (1990) Meth. Enzymol. 185:60-89) and the TAC promoter. Other promoters include, but are not limited to, the T7 phage promoter and other T7-like phage promoters, such as the T3, T5 and SP6 promoters, the trp, lpp, and lac promoters, such as the lacUV5, from E. coli; the P10 or polyhedrin gene promoter of baculovirus/insect cell expression systems (see, e.g, U.S. Pat. Nos. 5,243,041, 5,242,687, 5,266,317, 4,745,051, and 5,169,784) and inducible promoters from other eukaryotic expression systems.

Particularly preferred plasmids for transformation of E. coli cells include the pET expression vectors (see, U.S Pat. No. 4,952,496; available from NOVAGEN, Madison, Wis.; see, also literature published by Novagen describing the system). Such plasmids include pET 11a, which contains the T7lac promoter, T7 terminator, the inducible E. coli lac operator, and the lac repressor gene; pET 12a-c, which contains the T7 promoter, T7 terminator, and the E. coli ompT secretion signal; and pET 15b (NOVAGEN, Madison, Wis.), which contains a His-Tag™ leader sequence (Seq. ID NO. 23) for use in purification with a His column and a thrombin cleavage site that permits cleavage following purification over the column; the T7-lac promoter region and the T7 terminator.

Other preferred plasmids include the pKK plasmids, particularly pKK 223-3 (available from Pharmacia; see also, Brosius et al. (1984) Proc.. Nati. Acad. Sci. 81:6929; Ausubel et al., Current Protocols in Molecular Biology; U.S. Pat. Nos. 5,122,463, 5,173,403, 5,187,153, 5,204,254, 5,212,058, 5,212,286, 5,215,907, 5,220,013, 5,223,483, and 5,229,279), which contain the TAC promoter. Plasmid pKK has been modified by insertion of a kanamycin resistance cassette with EcoRI sticky ends (purchased from Pharmacia; obtained from pUC4K, see, em., Vieira et al. (1982) Gene 19:259-268; and U.S. Pat. No. 4,719,179) into the ampicillin resistance marker gene.

Other preferred vectors include the pP_{L}-lambda inducible expression vector and the tac promoter vector pDR450 (see, eg., U.S. Pat. Nos. 5,281,525, 5,262,309, 5,240,831, 5,231,008, 5,227,469, 5,227,293, available from Pharmacia P.L. Biochemicals, see; also Mott, et al. (1985) Proc. Natl. Acad. Sci. U.S.A. 82:88; and De Boer et al. (1983) Proc. Natl. Acad. Sci. U.S.A. 80:21); and baculovirus vectors, such as a pBlueBac vector (also called pJVETL and derivatives thereof; see, e.g., U.S. Pat. Nos. 5,278,050, 5,244,805, 5,243,041, 5,242,687, 5,266,317, 4,745,051, and 5,169,784), including pBlueBac III.

Other plasmids include the pIN-IllompA plasmids (see, U.S. Pat. No. 4,575,013 to Inouye; see, also, Duffaud et al. (1987) Meth. Enz. 153:492-507), such as pIN-IIIompA2 . The pIN-IIIompA plasmids include an insertion site for heterologous DNA linked in transcriptional reading frame with functional fragments derived from the lipoprotein gene of E. coli. The plasmids also include a DNA fragment coding for the signal peptide of the ompA protein of E. coli, positioned such that the desired polypeptide is expressed with the ompA signal peptide at its amino terminus, thereby allowing efficient secretion across the cytoplasmic membrane. The plasmids further include DNA encoding a specific segment of the E. coli lac promoter-operator, which is positioned in the proper orientation for transcriptional expression of the desired polypeptide, as well as a separate functional E. coli lacl gene encoding the associated repressor molecule that, in the absence of lac operon inducer, interacts with the lac promoter-operator to prevent transcription therefrom. Expression of the desired polypeptide is under the control of the lipoprotein (Ipp) promoter and the lac promoter-operator, although transcription from either promoter is normally blocked by the repressor molecule. The repressor is selectively inactivated by means of an inducer molecule thereby inducing transcriptional expression of the desired polypeptide from both promoters.

The repressor protein may be encoded by the plasmid containing the construct or a second plasmid that contains a gene encoding for a repressor-protein. The repressor-protein is capable of repressing the transcription of a promoter that contains sequences of nucleotides to which the repressor-protein binds. The promoter can be derepressed by altering the physiological conditions of the cell. The alteration can be accomplished by the addition to the growth medium of a molecule that inhibits, for example, the ability to interact with the operator or with regulatory proteins or other regions of the DNA or by altering the temperature of the growth media. Preferred repressor-proteins include, but are not limited to the E. coli lacl repressor responsive to IPTG induction, the temperature sensitive cl857 repressor. The E. coli lacl repressor is preferred.

In certain preferred embodiments, the constructs also include a transcription terminator sequence. The promoter regions and transcription terminators are each independently selected from the same or different genes. In some embodiments, the DNA fragment is replicated in bacterial cells, preferably in E. coli. The DNA fragment also typically includes a bacterial origin of replication, to ensure the maintenance of the DNA fragment from generation to generation of the bacteria. In this way, large quantities of the DNA fragment can be produced by replication in bacteria. Preferred bacterial origins of replication include, but are not limited to, the f1-ori and col E1 origins of replication.

Preferred bacterial hosts contain chromosomal copies of DNA encoding T7 RNA polymerase operably linked to an inducible promoter, such as the lacUV promoter (see, U.S. Pat. No. 4,952,496). Such hosts include, but are not limited to, lysogenic E. coli strains HMS174(DE3)pLysS, BL21 (DE3)pLysS, HMS174(DE3) and BL21 (DE3). Strain BL21 (DE3) is preferred. The pLys strains provide low levels of T7 ilysozyme, a natural inhibitor of T7 RNA polymerase. Preferred eukaryotic host are the insect cells Spodoptera frugiperda (sf9 cells; see, e.g., Luckow et al. (1988) Bio/technology 6:47-55 and U.S. Pat. No. 4,745,051).

For insect hosts, which are presently preferred, baculovirus vectors, such as a pIZ (see, em., U.S. Pat. Nos. 5,278,050, 5,244,805, 5,243,041, 5,242,687, 5,266,317, 4,745,051, and 5,169,784; available from INVITROGEN, San Diego) may also be used for expression of the polypeptides. The pBlueBacill vector is a dual promoter vector and provides for the selection of recombinants by blue/white screening as this plasmid contains the β-galactosidase gene (lacZ) under the control of the insect recognizable ETL promoter and is inducible with IPTG. A DNA construct is introduced into a baculovirus vector pBluebac III (INVITROGEN, San Diego, Calif.) and then co-transfected with wildtype virus into insect cells Spodoptera frugiperda (sf9 cells; see, e.g., Luckow et al. (1988) Bio/technology 6:47-55 and U.S. Pat. No. 4,745,051).

Other baculovirus vectors, such as pPbac and pMbac (available from Stratagene, San Diego, Calif., see, also Lernhardt et al. (1993) Strategies 6:20-21, and the Stratagene Catalog page 218), which contain the human alkaline phosphatase (see, e.g., Bailey et al. (1989) Proc. Natl. Acad. Sci. U. S. A. 86:22-26) and melittin (see, e.g., Tessier et al. (1991) Gene 98:177-183) secretory signals inserted into the BamHI and Ndel sites, respectively of pJVP10Z (see, e.g, Kawamoto et al. (1991) Biochem. Biophys. Res. Commun. 181:756-63, Ueda et al.(1994) Gene 140:267-272, are also suitable for use herein, particularly if secretion is desired. Insertion of genes into the Smal/BamHI sites of these vectors results in fusion proteins that are directed into the insect cell secretory pathway, which processes the pro-polypeptide so that mature peptide or fusion protein is secreted into the growth medium. Other heterologous signal sequences, such as the insulin signal sequence (see, em., U.S. Pat. No. 4,431,746 for DNA encoding the signal sequence), the growth hormone signal sequence, mammalian alkaline phosphatase, the mellitin signal sequence and others that are processed by insect cells are used.

The constructs provided herein have can be inserted into the baculovirus vector sold commercially under the name pBLUEBACIII (INVITROGEN, San Diego Calif.; see the INVITROGEN CATALOG; see, Vialard et al. (1990) J. Virol. 64:37; see also, U.S. Pat. Nos. 5,270,458; 5,243,041; and published International PCT Application WO 93/10139, which is based on U.S. patent application Ser. No. 07/792,600. The pBlueBaclll vector is a dual promoter vector and provides for the selection of recombinants by blue/white screening as this plasmid contains the β-galactosidase gene (lacZ) under the control of the insect recognizable ETL promoter and is inducible with IPTG. The construct of interest is inserted into this vector under control of the polyhedrin promoter. The DNA is then cotransfected, such as by Ca(PO₄)₂ or calcium phosphate transfection or liposomes, into Spodoptera frugiperda cells (sf9 cells) with wildtype baculovirus and grown in tissue culture flasks or in suspension cultures. Blue occlusion minus viral plaques are selected and plaque purified and screened for expression. Details are set forth in the Examples.

Combinatorial Methods

Molecules of desired specificity can be prepared using a variety of protocols in which portions of antibody molecules are combined to produce antigen binding molecules and are screened for desired specificity. Generally for phage display libraries, at least the Fab fragment is required, since secretion of the heterodimeric antibody fragment is involved.

Complementarily determining regions (CDRs) that are present in the Fab fragment can be varied by random mutagenesis (creation of “synthetic antibody libraries”) or by other methods. A phagemid vector such as pCombIII, designed to express the heavy chain antibody fragment as an N-terminal fusion with the pill phage coat protein domain (Barbas et al. (1991) Proc. Natl. Acad. Sci. USA, 88:7978) can be used for heterodimeric expression of the library of Fab antibody fragments (or Fab′2 antibody fragments) on the surface of phage, using bacteria.

Thus, for example, the DAV-1 heavy chain fused to the growth factor or a portion of the growth factor involved in binding to its receptor may be used to form the phage display library, and the library may then be “panned” for bifunctional antibodies that bind with high specificity to both the adenoviral surface protein and to the growth factor/cytokine receptor.

Linkers and Chemical Conjugation

For fusion protein the linkers are peptides; for chemically conjugated molecules the linkers may be other moieties. In addition, a combinations of chemically conjugated molecules and recombinantly produced portions, such as recombinantly produced antibodies or fragments or other antigen binding molecules may be combined and chemically fused to targeting agents, such as a growth factor or hormone. If a linker is used it is selected such that it does not interfere with the activity of the targeted agent upon interaction of the conjugate with a cell surface protein. Any appropriate linker known to those of skill in this art may be used. The linker may be selected to improve activity by permitting the targeted agent to complex with the viral or bacterial vector In some instances the linker is selected to increase the specificity, toxicity, solubility, serum stability, and/or intracellular availability targeted moiety. In some embodiments, several linkers may be included in order to take advantage of desired properties of each linker. Flexible linkers and linkers that increase solubility of the conjugates are contemplated for use, either alone or with other linkers are contemplated herein.

For chemical conjugation the components of the bifunctional molecules may be directly linked or attached via a linker. Linkers that are suitable for chemically linking conjugates include, but are not limited to, disulfide bonds, thioether bonds, hindered disulfide bonds, esters, and covalent bonds between free reactive groups, such as amine and thiol groups. These bonds are produced using heterobifunctional reagents to produce reactive thiol groups on one or both of the polypeptides and then reacting the thiol groups on one polypeptide with reactive thiol groups or amine groups on the other. Other linkers include, but are not limited to: acid cleavable linkers, such as bismaleimidoethoxy propane; acid labile-transferrin conjugates and adipic acid dihydrazide that are cleaved in more acidic environments; photocleavable cross linkers that are cleaved by visible or UV light.

Linkers that are suitable for chemically linked conjugates include, but are not limited to: disulfide bonds; thioether bonds; hindered disulfide bonds; and covalent bonds between free reactive groups, such as amine and thiol groups. These bonds are produced using heterobifunctional reagents to produce reactive thiol groups on one or both of the polypeptides and then reacting the thiol groups on one polypeptide with reactive thiol groups or amine groups to which reactive maleimido groups or thiol groups can be attached on the other. Other linkers include, acid cleavable linkers, such as bismaleimidoethoxy propane, acid labile-transferrin conjugates and adipic acid dihydrazide, that would be cleaved in more acidic intracellular compartments; cross linkers that are cleaved upon exposure to UV or visible light; and linkers, such as the various domains, such as C_{H}1, C_{H}2, and C_{H}3, from the constant region of human IgG₁ (see, Batra et al. (1993) Molecular Immunol. 30:379-386). In some embodiments, several linkers may be included in order to take advantage of desired properties of each linker.

Chemical linkers and peptide linkers may be inserted by covalently coupling the linker to the TA and the P portion (the αv integrin protein binding portion, such as an antibody). The heterobifunctional agents, described below, may be used to effect such covalent coupling. Peptide linkers may also be linked by expressing DNA encoding the linker and TA, linker and P, or linker, targeted agent and TA as a fusion protein.

Numerous heterobifunctional cross-linking reagents that are used to form covalent bonds between amino groups and thiol groups and to introduce thiol groups into proteins, are known to those of skill in this art (see, eg, the PIERCE CATALOG, ImmunoTechnology Catalog & Handbook, 1992-1993, which describes the preparation of and use of such reagents and provides a commercial source for such reagents; see, also, e.g., Cumber et al. (1992) Bioconjugate Chem. 3:397-401; Thorpe et al. (1987) Cancer Res. 47:5924-5931; Gordon et al. (1987) Proc. Natl. Acad Sci. 84:308-312; Walden et al. (1986) J. Mol. Cell Immunol. 2:191-197; Carlsson et al. (1978) Biochem. J. 173: 723-737; Mahan et al. (1987) Anal. Biochem. 162:163-170; Wawryznaczak et al. (1992) Br. J. Cancer 66:361-366; Fattom et al. (1992) Infection & Immun. 60:584-589). These reagents may be used to form covalent bonds between the TA and targeted agent. These reagents include, but are not limited to: N-succinimidyl-3-(2-pyridyldithio)propionate (SPDP; disulfide linker); sulfosuccinimidyl 6-[3-(2-pyridyldithio)propionamido]hexanoate (sulfo-LC-SPDP); succinimidyloxycarbonyl-α-methyl benzyl thiosulfate (SMBT, hindered disulfate linker); succinimidyl 6-[3-(2-pyridyidithio)propionamido]hexanoate (LC-SPDP); sulfosuccinimidyl 4-(N-maleimidomethyl)cyclo-hexane-1-carboxylate (sulfo-SMCC); succinimidyl 3-(2-pyridyidithio)butyrate (SPDB; hindered disulfide bond linker); sulfosuccinimidyl 2-(7-azido-4-methylcoumarin-3-acetamide)ethyl-1,3′-dithiopropionate (SAED); sulfo-succinimidyl 7-azido-4-methylcoumarin-3-acetate (SAMCA); sulfosuccinimidyl 6-[alpha-methyl-alpha-(2-pyridyldithio)toluamido]-hexanoate (sulfo-LC-SMPT); 1,4-di-[3′-(2′-pyridyldithio)propionamido]butane (DPDPB); 4-succinimidyloxycarbonyl-α-methyl-α-(2-pyridylthio)toluene (SMPT, hindered disulfate linker); sulfosuccinimidyl6[α-methyl-α-(2-pyridyidithio)toluamido]hexanoate (sulfo-LC-SMPT); m-maleimidobenzoyl-N-hydroxysuccinimide ester (MBS); m-maleimidobenzoyl-N-hydroxysulfosuccinimide ester (sulfo-M BS); N-succinimidyl (4-iodoacetyl)aminobenzoate (SIAB; thioether linker); sulfosuccinimidyl(4-iodoacetyl)amino benzoate (sulfo-SIAB); succinimidyl 4(p-maleimido-phenyl)butyrate (SMPB); sulfosuccinimidyl 4-(p-maleimidophenyl)butyrate (sulfo-SMPB); azidobenzoyl hydrazide (ABH).

Acid cleavable linkers, photocleavable and heat sensitive linkers may also be used, particularly where it may be necessary to cleave the targeted agent to permit it to be more readily accessible to reaction. Acid cleavable linkers include, but are not limited to, bismaleimidoethoxy propane; and adipic acid dihydrazide linkers (see, e.g., Fattom et al. (1992) Infection & Immun. 60:584-589) and acid labile transferrin conjugates that contain a sufficient portion of transferrin to permit entry into the intracellular transferrin cycling pathway (see, e.g., Welhöner et al. (1991) J. Biol. Chem. 266:4309-4314).

Photocleavable linkers are linkers that are cleaved upon exposure to light (see, e.g., Goldmacher et al. (1992) Bioconi. Chem. 3:104-107, which linkers are herein incorporated by reference), thereby releasing the targeting agent from the bifunctional molecule and the complex upon exposure to light. Photocleavable linkers that are cleaved upon exposure to light are known (see, e.g., Hazum et al. (1981) in Pept., Proc. Eur. Pept. Symp., 16th, Brunfeldt, K (Ed), pp. 105-110, which describes the use of a nitrobenzyl group as a photocleavable protective group for cysteine; Yen et al. (1989) Makromol. Chem 190:69-82, which describes water soluble photocleavable copolymers, including hydroxypropylmethacrylamide copolymer, glycine copolymer, fluorescein copolymer and methylrhodamine copolymer; Goldmacher et al. (1992) Bioconj. Chem. 3:104-107, which describes a cross-linker and reagent that undergoes photolytic degradation upon exposure to near UV light (350 nm); and Senter et al. (1985) Photochem. Photobiol 42:231-237, which describes nitrobenzyloxycarbonyl chloride cross linking reagents that produce photocleavable linkages), thereby releasing the targeted agent upon exposure to light. Such linkers would have particular use in treating dermatological or ophthalmic conditions that can be exposed to light using fiber optics. After administration of the conjugate, the eye or skin or other body part can be exposed to light, resulting in release of the targeted moiety from the conjugate. Such photocleavable linkers are useful in connection with diagnostic protocols in which it is desirable to remove the targeting agent to permit rapid clearance from the body of the animal.

3. Preparation of Complexes of the Bifunctional Molecules with Viral Vector Particles or Bacterial Particles

The complexes between the bifunctional molecules and vectors can be prepared by incubating the vectors and bifunctional molecules under suitable conditions to effect formation of the complexes.

Assays for Activity

Any assay to assess the ability of the resulting complexes to deliver genes. Such assays are known to those of skill in the art, and several are exemplified herein (see EXAMPLES).

4. Exemplary Embodiment DAV-1 Constructs and use Thereof for Targeting

Exemplary of the embodiments contemplated herein are bifunctional molecules formed by linkage of a targeting agent, such as a growth factor known to bind to receptors that activate PI3K, to an antibody or antigen-binding portion thereof, that specifically binds to the penton protein of a variety of adenovirus serotypes. It is understood that these embodiments are exemplary and that any antibody that binds to a protein on a viral particle or bacterial cell or other moiety intended for delivery into a cell can be used to specifically target such moieties to cells by virtue of interaction of the targeting agent with cell surface receptors. Presently preferred are adenovirus particles and the penton protein thereof that is responsible for interaction with αv integrins, which promote viral internalization.

A penton base monoclonal antibody, DAV-1, which recognizes the integrin binding site on adenovirus particle types 2/5 (Stewart et al. (1997) EMBO J. 16:1189-1198; EXAMPLE 1), binds to the penton base with high affinity but does not inhibit virus infection is re-engineered herein (see, EXAMPLE 1). Nucleic acid encoding the DAV-1 heavy chain (see, e.g., SEQ ID Nos. 5) has been fused with one of several different cytokines/growth factors known to activate PI3K and the encoded fusion proteins are co-expressed in insect cells with the DAV-1 light chain. The resulting bifunctional molecules retain immunoreactivity and cytokine function.

The bifunctional molecules have been complexed with adenovirus and shown to provide increased gene delivery to cells lacking αv integrins, as well as cells expressing αv integrins, evidencing PI3K activation. Complexing with the bifunctional molecule also allows gene delivery by a fiberless Ad vector that lacks the ability to bind CAR (see, EXAMPLES 5 and 6).

a. Analysis of Bifunctional Signaling Antibodies

DAV-1 bifunctional signaling-antibodies, designated DT (DAV-1 fused to TNF-α), DI (DAV-1 fused to IGF-1), and DE (DAV-1 fused to EGF), were expressed in insect cells as secreted proteins and purified on Protein L affinity columns. The DT heavy chain (D_{H}T) had an apparent molecular weight of approximately 70 kDa, consistent with the combined sizes of the DAV-1γ heavy chain (53 kDa) and monomeric TNF-α ligand (17 kDA). The apparent molecular weight of the κ light chains of DAV-1 (D_{L}) was identical to that of the recombinant DT molecule (approx. 25 kDa). Western blot analyses showed that the DAV-1 mAb (D) and the DT molecules) were recognized by an anti-mouse IgG antibody, while only the DT molecule was recognized by an anti-TNF-α polyclonal antibody.

DT molecules were capable of binding to immobilized penton base or Ad particles in an ELISA and elicited cytotoxicity against a TNF-α sensitive cell line, MCF-7, indicating that the DT bifunctional molecule retains virus and cytokine receptor binding functions.

b. Bifunctional Molecules Promote Ad-mediated Gene Delivery to Cells Lacking αv Integrins

A first generation adenovirus vector containing a RSV-driven LacZ reporter gene with DT was preincubated at a ratio of 2 antibody molecules per RGD motif. This complex was then added to M21-L12 human melanoma cells, which do not express αv integrins (Felding-Habermann et al. (1992) J. Clin. Invest. 89:2018-2022), but can support efficient virus binding (Wickham et al. (1993) Cell 73:309-319). Ad complexed with DT but not D alone, significantly increases Ad-mediated gene delivery to M21-L12 cells as measured by transgene expression at 48 hrs post-infection. Approximately 60% of cells incubated with Ad plus DT stained positive for β-galactosidase, compared to less than 3% of calls that had been incubated with virus alone or virus plus D. The increase in gene delivery by DT was not due to increased activation of the RSV LTR transgene promoter as a consequence of ligation of the TNF receptor, since M21-L12 cells that had been infected with adenovirus alone for three hours followed by addition of DT showed very little increase in gene delivery at 48 hours postinfection. This result indicates that bifunctional molecules increase adenovirus-mediated gene delivery by enhancing one or more steps associated with cell entry.

C. DT Molecules Enhance Ad Binding and Internalization

The following experiments showed that DT enhancement of gene delivery was associated with increased virus attachment to M21-L12 cells. Pre-incubation of ¹²⁵I-labeled Ad particles with DT but not D molecules increased virus binding approximately 5 fold. To investigate the molecules responsible for increased binding, competition experiments were performed. Ad-DT binding to cells was measured in the presence of a 50-fold excess of recombinant fiber protein or anti-TNF-α or a combination of these molecules. Either recombinant fiber or anti-TNF-α antibody alone was capable of blocking only 20-25% of Ad-DT binding to cells. In contrast, approximately 70% of binding could be inhibited by a combination of fiber and anti-TNF-α. These findings indicate that Ad-DT -binding to cells is mediated by CAR-fiber interaction as well as TNF-α-receptor association.

d. DT Molecules Potentiate Internalization of ¹²⁵I-labeled Virus Particles as Measured by Resistance to Trypsin Digestion.

As demonstrated in earlier studies(Wickham et al. (1993) Cell 73:309-319), M21-L12 cells support relatively low levels of adenovirus internalization. This is due to the absence of αv integrins. DT molecules significantly increased the rate and extent of adenovirus internalization into these cells. Together, these findings indicate that DT molecules enhance gene delivery by promoting virus binding as well as virus internalization.

e. DT Enhancement of Gene Delivery is Associated with PI3K Activation

Efficient Ad internalization via αv integrins requires activation of PI3K, a key cellular signaling molecule. M21-L12 cells were treated with PI3K inhibitors, wortmannin or LY292004 prior to virus infection to show that DT enhancement of gene delivery also involves PI3K,. Wortmannin and LY294002 inhibited Ad-mediated gene delivery by approximately 70% and 50%, respectively, indicating that PI3K activity plays a major role in DT enhancement of gene delivery.

For further demonstration of the role of PI3K-dependent signaling in enhanced gene delivery, Ad-mediated gene delivery by other bifunctional molecules whose cytokine/growth factor domains are known to activate PI3K was measured. DT, DE and DI molecules enhanced gene delivery by approximately 30, 10 and 5 fold respectively. Enhanced gene delivery by these molecules was also inhibited by pretreatment of cells with wortmannin. These findings further demonstrate that PI3K activation promotes Ad gene delivery.

f. Bifunctional Molecules Allow Gene Delivery by Fiberless Adenovirus Particles

Fiberless adenovirus vectors that cannot bind to CAR have been constructed (see, e.g., copending U.S. application Ser. No. 09/482,682 and U.S. application Ser. No. 09/562,934; see, also Von Seggern et al. (1999) J. Virol. 73:1601-1608). The structure of these particles is nearly identical to that of wildtype virions. Fiberless particles alone showed almost no transgene delivery to SW480 epithelial cells, even though these cells express both CAR and integrin αvβ5 (Von Seggern et al. (1999) J. Virol. 73:1601-1608).

DT molecules enhanced gene delivery of fiberless viruses. Fiberless particles complexed with DT or DI molecules exhibited increased gene delivery approximately 10-15, 3 and 5 fold, respectively, compared to the uncomplexed fiberless particles. These findings indicate that fiberless particles can be retargeted to cells via signal transducing antibodies. The use of fiberless adenoviral vectors for gene therapy is desirable because it is then possible to restrict viral tropism to selected cell types by adopting a specific targeting strategy and abrogating the interaction between the viral fiber protein and the CAR receptors.

g. Summary of Results

It is demonstrated herein that Ad vectors complexed with bifunctional molecules significantly increased gene delivery to human melanoma cells lacking αv integrins and was associated with both increased virus binding and internalization compared to Ad vectors without such bifuctional molecules. Importantly, PI3K activation plays a key role in this process as a significant reduction in delivery following treatment of cells with pharmacologic inhibitors of PI3K was observed. Moreover, bifunctional molecules displaying distinct growth factor ligands, each of which is known to activate PI3K, also enhanced gene delivery. The variability in gene delivery by different bifunctional molecules is most likely to be due to differences in the expression of growth factor/cytokine receptors on different cell types or to the extent of PI3K activation induced by each ligand.

Achieving gene delivery to specific cell types has been hampered by the fiber receptor (CAR), which is expressed on multiple cell types. To circumvent this problem, a fiberless adenovirus vector was retargeted with bifunctional molecules. Significant enhancement of gene delivery by a fiberless adenovirus complexed with several different bifunctional molecules was detected. These studies confirm the structural and functional integrity of fiberless adenovirus particles and indicate their usefulness for retargeting in gene delivery.

The PI3K-dependent signaling pathway should be useful for increasing the uptake of other viral vectors. For example, adeno-associated virus (AAV) has also been reported to use αv integrins for infection. Because a variety of bacterial and viral pathogens use integrins and/or PI3K activation for host cell invasion indicates that targeting of PI3K-dependent signaling pathways can also be used as a general scheme for potentiating cell entry of nonviral vectors. The exemplified methods and bifunctional molecules can be adapted for use for potentiating entry of other viral vectors and bacterial vectors that use the PI3K-dependent signaling pathways for internalization.

C. Construction of the Viral Particles

1. Selection of Viral Genome and Fiber Protein

Methods for preparing recombinant adenoviral vectors for gene product delivery are well known. Preferred among those are the methods exemplified herein (see EXAMPLES) and also described in copending U.S. application Ser. No. 09/482,682 (also filed as International PCT application No. PCT/US00/00265, filed Jan. 14, 2000, which claims priority to U.S. provisional application Serial No. 60/115,920, as does U.S. application Ser. No. 09/482,682)).

As noted, any desired recombinant adenovirus is contemplated for use in the methods herein as long as the viral genome is packaged in a capsid that includes at least the portion of a fiber protein that provides selective binding to photoreceptor cells. This fiber protein is preferably from an adenovirus type D serotype and is preferably an Ad37 fiber. The fiber protein should retain the knob region at the C-terminus (“head domain”) from the Ad type D virus that contains the type-specific antigen and is responsible for binding to the cell surface receptor. Hence the fiber protein can be a chimeric fiber protein as long as it retains a sufficient portion of the type D serotype to specifically bind to photoreceptor cells. Generally the portion retained will be all or a portion of the knob region. The precise amount of knob region required can be determined empirically by including portions thereof and identifying the minimum residues from and Ad type D serotype, preferably Ad37, to effect selective targeting of a virion packaged with such fiber to photoreceptors in the eye upon introduction of the packaged virion into the aqueous humor.

Recombinant adenovirus containing heterologous nucleic acids that encode a desired product, such a gene to correct a genetic defect, may be made by any methods known to those of skill in the art. The viruses are packaged in a suitable cell line.

The family of Adenoviridae includes many members with at least 51 known serotypes of human adenovirus (Ad1-Ad47) (Shenk, Virology, Chapter 67, in Fields et al., eds. Lippincott-Raven, Philadelphia, 1996,) as well as members of the genus Mastadenovirus including human, simian, bovine, equine, porcine, ovine, canine and opossum viruses, and members of the Aviadenovirus genus, including bird viruses, e.g. CELO. Thus it is contemplated that the methods herein can be applied to any recombinant viral vectors derived from any adenovirus species. One of skill in the art would have knowledge of the different adenoviruses (see, e.g.,Shenk, Virology, Chapter 67, in Fields et al., eds. Lippincott-Raven, Philadelphia, 1996,) and can construct recombinant viruses containing portions of the genome of any such virus. The methods herein may also be adapted for use with any delivery vehicle that internalizes via receptors that use the PI3K signalling pathway, particularly that employ αv integrins in the process.

2. Packaging

Recombinant adenoviral vectors generally have at least a deletion in the first viral early gene region, referred to as E1, which includes the E1a and E1b regions. Deletion of the viral E1 region renders the recombinant adenovirus defective for replication and incapable of producing infectious viral particles in subsequently-infected target cells. Thus, to generate E1-deleted adenovirus genome replication and to produce virus particles requires a system of complementation which provides the missing E1 gene product. E1 complementation is typically provided by a cell line expressing E1, such as the human embryonic kidney packaging cell line, i.e. an epithelial cell line, called 293. Cell line 293 contains the E1 region of adenovirus, which provides E1 gene region products to “support” the growth of E1-deleted virus in the cell line (see, e.g., Graham et al., J. Gen. Virol. 36: 59-71, 1977). Additionally, cell lines that may be usable for production of defective adenovirus having a portion of the adenovirus E4 region have been reported (WO 96/22378). Multiply deficient adenoviral vectors and complementing cell lines have also been described (WO 95/34671, U.S. Pat. No. 5,994,106).

Copending U.S. application Ser. No. 09/482,682 (also filed as International PCT application No. PCT/US00/00265, filed Jan. 14, 2000)) provides packaging cell lines that support viral vectors with deletions of major portions of the viral genome, without the need for helper viruses and also provides cell lines and helper viruses for use with helper-dependent vectors.

The copending application provides a packaging cell line that has heterologous DNA stably integrated into the chromosomes of the cellular genome. The heterologous DNA sequence encodes one or more adenovirus regulatory and/or structural polypeptides that complement the genes deleted or mutated in the adenovirus vector genome to be replicated and packaged. The packaging cell line expresses, for example, one or more adenovirus structural proteins, polypeptides, or fragments thereof, such as penton base, hexon, fiber, polypeptide IIIa, polypeptide V, polypeptide VI, polypeptide VII, polypeptide VIII, and biologically active fragments thereof. The expression can be constitutive or under the control of a regulatable promoter. These cell lines are particularly designed for expression of recombinant adenoviruses intended for delivery of therapeutic products.

Particular packaging cell lines complement viral vectors having a deletion or mutation of a DNA sequence encoding an adenovirus structural protein, regulatory polypeptides E1A and E1B, and/or one or more of the following regulatory proteins or polypeptides: E2A, E2B, E3, E4, L4, or fragments thereof.

The packaging cell lines are produced by introducing each DNA molecule into the cells and then into the genome via a separate complementing plasmid or plurality of DNA molecules encoding the complementing proteins can be introduced via a single complementing plasmid.

For therapeutic applications, the delivery plasmid further includes a nucleotide sequence encoding a foreign polypeptide. Exemplary delivery plasmids is pDV44, pE1B gal and pE1sp1B. In a similar or analogous manner, therapeutic genes may be introduced.

The cell further includes a complementing plasmid encoding a fiber as contemplated herein; the plasmid or portion thereof is integrated into a chromosome(s) of the cellular genome of the cell.

In one embodiment, a composition comprises a cell containing first and second delivery plasmids wherein a first delivery plasmid comprises an adenovirus genome lacking a nucleotide sequence encoding fiber and incapable of directing the packaging of new viral particles in the absence of a second delivery plasmid, and a second delivery plasmid comprises an adenoviral genome capable of directing the packaging of new viral particles in the presence of the first delivery plasmid.

The packaging cell line can be derived from a procaryotic cell line or from a eukaryotic cell line. While various embodiments suggest the use of mammalian cells, and more particularly, epithelial cell lines, a variety of other, non-epithelial cell lines are used in various embodiments.

3. Components of the Nucleic Acid Molecule Included in the Particle

A recombinant viral vector or therapeutic viral vector for use in the methods herein, typically includes a nucleic acid fragment that encodes a protein or polypeptide molecule, or a biologically active fragment thereof, or other regulatory sequence, that is intended for use for therapeutic applications.

The nucleic acid molecule to be packaged in the viral particle also may include an enhancer element and/or a promoter located 3′ or 5′ to and controlling the expression of the therapeutic product-encoding nucleic acid moleucle if the product is a protein. Further, for purposes herein, the promoter and/or other transcriptional and translational regulatory sequences controlling expression of the product is preferably one that is expressed specifically in the targeted cells, such as the a photoreceptor-specific promoter, such as a rhodopsin gene promoter.

The nucleic acid molecule to be packaged in viral capsid includes at least 2 different operatively linked DNA segments. The DNA can be manipulated and amplified by PCR as described herein and by using standard techniques, such as those described in Molecular Cloning: A Laboratory Manual, 2nd Ed., Sambrook et al., eds., Cold Spring Harbor, New York (1989). Typically, to produce such molecule, the sequence encoding the selected polypeptide and the promoter or enhancer are operatively linked to a DNA molecule capable of autonomous replication in a cell either in vivo or in vitro. By operatively linking the enhancer element or promoter and nucleic acid molecule to the vector, the attached segments are replicated along with the vector sequences.

Thus, the recombinant DNA molecule (rDNA) is a hybrid DNA molecule comprising at least 2 nucleotide sequences not normally found together in nature. In various preferred embodiments, one of the sequences is a sequence encoding an Ad-derived polypeptide, protein, or fragment thereof. The nucleic acid molecule intended to be packaged is from about 20 base pairs to about 40,000 base pairs in length, preferably about 50 bp to about 38,000 bp in length. In various embodiments, the nucleic acid molecule is of sufficient length to encode one or more adenovirus proteins or functional polypeptide portions thereof. Since individual Ad polypeptides vary in length from about 19 amino acid residues to about 967 amino acid residues, encoding nucleic acid molecules from about 50 bp up to about 3000 bp, depending on the number and size of individual polypeptide-encoding sequences that are “replaced” in the viral vectors by therapeutic product-encoding nucleic acid molecules.

Preferably the molecule includes an adenovirus tripartite leader (TPL) nucleic acid sequence operatively linked to an intron containing RNA processing signals (such as for example, splice donor or splice acceptor sites) suitable for expression in the packaging cell line. Most preferably the intron contains a splice donor site and a splice acceptor site. Alternatively, the TPL nucleotide sequence may not comprise an intron. The intron includes any sequence of nucleotides that function in the packaging cell line to provide RNA processing signals, including splicing signals. Introns have been well characterized from a large number of structural genes, and include but are not limited to a native intron 1 from adenovirus, such as Ad5's TPL intron 1; others include the SV40 VP intron; the rabbit beta-globin intron, and synthetic intron constructs (see, e.g., Petitclerc et al. (1995)J. Biothechnol., 40:169; and Choi et al. (1991 Mol. Cell. Biol., 11:3070).

The nucleic acid molecule encoding the TPL includes either (a) first and second TPL exons or (b) first, second and third TPL exons, where each TPL exon in the sequence is selected from among the complete TPL exon 1, partial TPL exon 1, complete TPL exon 2 and complete TPL exon 3. A complete exon is one which contains the complete nucleic acid sequence based on the sequence found in the wildtype viral genome. Preferably the TPL exons are from Ad2, Ad3, Ad5, Ad7 and the like, however, they may come from any Ad serotype, as described herein. A preferred partial TPL exon 1 is described in the Examples. The use of a TPL with a partial exon 1 has been reported (International PCT application No. WO 98/13499).

The intron and the TPL exons can be operatively linked in a variety of configurations to provide a functional TPL nucleotide sequence, An intron may not be a part of the construct. For example, the intron can be positioned between any of TPL exons 1, 2 or 3, and the exons can be in any order of first and second, or first/second/third. The intron can also be placed preceding the first TPL exon or following the last TPL exon. In a preferred embodiment, complete TPL exon 1 is operatively linked to complete TPL exon 2 operatively linked to complete TPL exon 3. In a preferred variation, adenovirus TPL intron 1 is positioned between complete TPL exon 1 and complete TPL exon 2. It may also be possible to use analogous translational regulators from other viral systems such as rabiesvirus.

4. Complementing Plasmids

Also contemplated are the use of nucleic acid molecules, typically in the form of DNA plasmid vectors, which are capable of expression of an adenovirus structural protein or regulatory protein. Because these expression plasmids are used to complement the defective genes of a recombinant adenovirus vector genome, the plasmids are referred to as complementing or complementation plasmids.

The complementing plasmid contains an expression cassette, a nucleotide sequence capable of expressing a protein product encoded by the nucleic acid molecule. Expression cassettes typically contain a promoter and a structural gene operatively linked to the promoter. The complementing plasmid can further include a sequence of nucleotides encoding TPL nucleotide to enhance expression of the structural gene product when used in the context of adenovirus genome replication and packaging.

A complementing plasmid can include a promoter operatively linked to a sequence of nucleotides encoding an adenovirus structural polypeptide, such as, but are not limited to, penton base; hexon; fiber; polypeptide IIIa; polypeptide V; polypeptide VI; polypeptide VII; polypeptide VIII; and biologically active fragments thereof. In another variation, a complementing plasmid may also include a sequence of nucleotides encoding a first adenovirus regulatory polypeptide, a second regulatory polypeptide, and/or a third regulatory polypeptide; or any combination of the foregoing.

5. Nucleic Acid Molecule Synthesis

A nucleic acid molecule comprising synthetic oligonucleotides can be prepared using any suitable method, such as, the phosphotriester or phosphodiester methods (see, e.g., Narang (1979) et al., Meth. Enzymol., 68:90; U.S. Pat. No. 4,356,270; and Brown et al., (1979) Meth. Enzymol., 68:109). For oligonucleotides, the synthesis of the family members can be conducted simultaneously in a single reaction vessel, or can be synthesized independently and later admixed in preselected molar ratios. For simultaneous synthesis, the nucleotide residues that are conserved at preselected positions of the sequence of the family member can be introduced in a chemical synthesis protocol simultaneously to the variants by the addition of a single preselected nucleotide precursor to the solid phase oligonucleotide reaction admixture when that position number of the oligonucleotide is being chemically added to the growing oligonucleotide polymer. The addition of nucleotide residues to those positions in the sequence that vary can be introduced simultaneously by the addition of amounts, preferably equimolar amounts, of multiple preselected nucleotide precursors to the solid phase oligonucleotide reaction admixture during chemical synthesis. For example, where all four possible natural nucleotides (A, T, G and C) are to be added at a preselected position, their precursors are added to the oligonucleotide synthesis reaction at that step to simultaneously form four variants (see, e.g., Ausubel et al. (Current Protocols in Molecular Biology, Suppl. 8. p.2.11.7, John Wiley & Sons, Inc., New York, 1991).

Nucleotide bases other than the common four nucleotides (A, T, G or C), or the RNA equivalent nucleotide uracil (U), can also be used. For example, it is well known that inosine (I) is capable of hybridizing with A, T and G, but not C. Examples of other useful nucleotide analogs are known in the art and may be found referred to in 37 C.F.R. §1.822.

Thus, where all four common nucleotides are to occupy a single position of a family of oligonucleotides, that is, where the preselected nucleotide sequence is designed to contain oligonucleotides that can hybridize to four sequences that vary at one position, several different oligonucleotide structures are contemplated. The composition can contain four members, where a preselected position contains A, T, G or C. Alternatively, a composition can contain two nucleotide sequence members, where a preselected position contains I or C, and has the capacity the hybridize at that position to all four possible common nucleotides. Finally, other nucleotides may be included at the preselected position that have the capacity to hybridize in a non-destabilizing manner with more than one of the common nucleotides in a manner similar to inosine.

Similarly, larger nucleic acid molecules can be constructed in synthetic oligonucleotide pieces, and assembled by complementary hybridization and ligation, as is well known.

D. Adenovirus Expression Vector Systems

The adenovirus vector genome that is encapsulated in the virus particle and that expresses exogenous genes in a gene therapy setting is a key component of the system, which systems are well known and readily available. Thus, the components of a recombinant adenovirus vector genome include the ability to express selected adenovirus structural genes, to express a desired exogenous protein, and to contain sufficient replication and packaging signals that the genome is packaged into a gene delivery vector particle. The preferred replication signal is an adenovirus inverted terminal repeat containing an adenovirus origin of replication, as is well known.

Although adenovirus include many proteins, not all adenovirus proteins are required for assembly of a recombinant adenovirus particle (vector). Thus, deletion of the appropriate genes from a recombinant Ad vector permits accommodation of even larger “foreign” DNA segments.

Particularly contemplated are helper dependent systems as described above, in which the adenovirus vector genome does not encode a functional adenovirus fiber protein. A non-functional fiber gene refers to a deletion, mutation or other modification to the adenovirus fiber gene such that the gene does not express any or insufficient adenovirus fiber protein to package a fiber-containing adenovirus particle without complementation of the fiber gene by a complementing plasmid or packaging cell line. Such a genome is referred to as a “fiberless” genome, not to be confused with a fiberless particle. Alternatively, a fiber protein may be encoded but is insufficiently expressed to result in a fiber containing particle.

Thus, among the delivery vectors contemplated for use are helper-independent fiberless recombinant adenovirus vector genomes that include genes that (a) express all adenovirus structural gene products but express insufficient adenovirus fiber protein to package a fiber-containing adenovirus particle without complementation of said fiber gene, (b) express an exogenous protein, and (c) contains an adenovirus packaging signal and inverted terminal repeats containing adenovirus origin of replication.

The adenovirus vector genome is propagated in the laboratory in the form of rDNA plasmids containing the genome, and upon introduction into an appropriate host, the viral genetic elements provide for viral genome replication and packaging rather than plasmid-based propagation. Exemplary methods for preparing an Ad-vector genome are described in the Examples.

A vector herein includes a nucleic acid (preferably DNA) molecule capable of autonomous replication in a cell and to which a DNA segment, e.g., a gene or polynucleotide, can be operatively linked to bring about replication of the attached segment. For purposes herein, one of the nucleotide segments to be operatively linked to vector sequences encodes at least a portion of a therapeutic nucleic acid molecule. As noted above, therapeutic nucleic acid molecules include those encoding proteins and also those that encode regulatory factors that can lead to expression or inhibition or alteration of expression of a gene product in a targeted cell.

1. Nucleic Acid Gene Expression Cassettes

In various embodiments, a peptide-coding sequence of the therapeutic gene is inserted into an expression vector and expressed; however, it is also feasible to construct an expression vector which also includes some non-coding sequences as well. Preferably, however, non-coding sequences are excluded. Alternatively, a nucleotide sequence for a soluble form of a polypeptide may be utilized. Another preferred therapeutic viral vector includes a nucleotide sequence encoding at least a portion of a therapeutic nucleotide sequence operatively linked to the expression vector for expression of the coding sequence in the therapeutic nucleotide sequence.

The choice of viral vector into which a therapeutic nucleic acid molecule is operatively linked depends directly, as is well known in the art, on the functional properties desired, e.g., vector replication and protein expression, and the host cell to be transformed. Although certain adenovirus serotypes are recited herein in the form of specific examples, it should be understood that the use of any adenovirus serotype, including hybrids and derivatives thereof are contemplated.

A translatable nucleotide sequence is a linear series of nucleotides that provide an uninterrupted series of at least 8 codons that encode a polypeptide in one reading frame. Preferably, the nucleotide sequence is a DNA sequence. The vector itself may be of any suitable type, such as a viral vector (RNA or DNA), naked straight-chain or circular DNA, or a vesicle or envelope containing the nucleic acid material and any polypeptides that are to be inserted into the cell.

2. Promoters

As noted elsewhere herein, an expression nucleic acid in an Ad-derived vector may also include a promoter, particularly a tissue or cell specific promoter, preferably one expressed in the targeted cells.

Promoters nucleic acid fragments that contain a DNA sequence that controls the expression of a gene located 3′ or downstream of the promoter. The promoter is the DNA sequence to which RNA polymerase specifically binds and initiates RNA synthesis (transcription) of that gene, typically located 3′ of the promoter. A promoter also includes DNA sequences which direct the initiation of transcription, including those to which RNA polymerase specifically binds. If more than one nucleic acid sequence encoding a particular polypeptide or protein is included in a therapeutic viral vector or nucleotide sequence, more than one promoter or enhancer element may be included, particularly if that would enhance efficiency of expression. Regulatable (inducible) as well as constitutive promoters may be used, either on separate vectors or on the same vector. For example, some useful regulatable promoters are those of the CREB-regulated gene family and include inhibin, gonadotropin, cytochrome c, glucagon, and the like. (See, e.g., International PCT application No. No. WO 96/14061). Preferably the promoter selected is from a photoreceptor-specific gene, such as a rhodopsin gene or gene that encodes a protein that regulates rhodopsin expression.

A regulatable or inducible promoter may be described as a promoter wherein the rate of RNA polymerase binding and initiation is modulated by external stimuli. (see, e.g., U.S. Pat. Nos. 5,750,396 and 5,998,205). Such stimuli include various compounds or compositions, light, heat, stress, chemical energy sources, and the like. Inducible, suppressible and repressible promoters are considered regulatable promoters.

Regulatable promoters may also include tissue-specific promoters. Tissue-specific promoters direct the expression of the gene to which they are operably linked to a specific cell type. Tissue-specific promoters cause the gene located 3′ of it to be expressed predominantly, if not exclusively, in the specific cells where the promoter expressed its endogenous gene. Typically, it appears that if a tissue-specific promoter expresses the gene located 3′ of it at all, then it is expressed appropriately in the correct cell types (see, e.g., Palmiter et al. (1986) Ann. Rev. Genet. 20: 465-499).

E. Formulation and Administration

Compositions containing therapeutically effective concentrations of recombinant adenovirus delivery vectors for delivery of therapeutic gene products to cells that express the targeted receptor, such as IGF-1 receptors and TNF-α receptors.

Preferable modes of administration include, local and topical modes of administration, such as, but are not limited to, intramuscular, intravenous, intraperitoneal and subretinal injection, particularly intravitreal injection,

The recombinant viral compositions may also be formulated for implantation into tissues, including as the anterior or posterior chamber of the eye, particularly the vitreous cavity, in sustained released formulations, such as adsorbed to biodegradable supports, including collagen sponges, or in liposomes. Sustained release formulations may be formulated for multiple dosage administration, so that during a selected period of time, such as a month or up to about a year, several dosages are administered. Thus, for example, liposomes may be prepared such that a total of about two to up to about five or more times the single dosage is administered in one injection. The vectors are formulated in an pharmaceutically acceptable carriers for the selected route of administration in a volume suitable for such route.

To prepare compositions the viral particles are dialyzed into a suitable pharmaceutically acceptable carrier or viral particles may be concentrated and/or mixed therewith. The resulting mixture may be a solution, suspension or emulsion. In addition, the viral particles may be formulated as the sole pharmaceutically active ingredient in the composition or may be combined with other active agents for the particular disorder treated.

Suitable carriers include, but are not limited to, physiological saline, phosphate buffered saline (PBS), balanced salt solution (BSS), lactate Ringers solution, and solutions containing thickening and solubilizing agents, such as glucose, polyethylene glycol, and polypropylene glycol and mixtures thereof. Liposomal suspensions may also be suitable as pharmaceutically acceptable carriers. These may be prepared according to methods known to those skilled in the art.

The compositions can be prepared with carriers that protect them against rapid elimination from the body, such as time release formulations or coatings. Such carriers include controlled release formulations, such as, but not limited to, microencapsulated delivery systems, and biodegradable, biocompatible polymers, such as ethylene vinyl acetate, polyanhydrides, polyglycolic acid, polyorthoesters, polylactic acid and other types of implants that may be placed directly into the body or tissue of interest.

Liposomal suspensions may also be suitable as pharmaceutically acceptable carriers. Preferably such liposomes. For example, liposome formulations may be prepared by methods known to those of skill in the art [see, e., Kimm et al. (1983) Bioch. Bioph. Acta 728:339-398; Assil et al. (1987) Arch Ophthalmol. 105:400; and U.S. Pat. No. 4,522,811]. The viral particles may be encapsulated into the aqueous phase of liposome systems. The active materials can also be mixed with other active materials, that do not impair the desired action, or with materials that supplement the desired action or have other action, such as anti-tumor agents.

For purposes herein, the viral or other particles may be complexed with the bifunctional molecules (the conjugates) prior to packaging or immediately prior to use. Hence combinations and kits containing the combinations of the selected delivery vector and the bifunctional molecules are also provided. The bifunctional molecules and delivery vectors may be packaged as separate compositions or as a single composition. The kits optionally include instructions for use and administration of the combinations.

The compositions can be enclosed in ampules, disposable syringes or multiple or single dose vials made of glass, plastic or other suitable material. Such enclosed compositions can be provided in kits. In particular, kits containing vials, ampules or other container, preferably disposable vials or containers or other packages with sufficient amount of the composition to deliver a desired amount, which depends upon the treated condition.

Finally, the combinations and components compositions thereof may be packaged as articles of manufacture containing packaging material, typically a vial or container, an pharmaceutically acceptable composition containing the viral particles and a label that indicates the therapeutic use of the composition.

Also provided are kits for practice of the methods herein. The kits contain one or more containers, such as sealed vials, with sufficient composition for single dosage administration.

Administration

The compositions containing the compounds are administered systemically by any suitable route, or may be administered topically, such as by injection into synovial fluids for treatment of rheumatoid arthritis, in the form of penetrating eyedrops for treatment of occular disorders or disorders in which the vectors can be suitably targeted from the eye.

It is further understood that, for any particular subject and disorder, specific dosage regimens should be adjusted over time according to the individual need and the professional judgment of the person administering or supervising the administration of the recombinant viruses, and that the concentration ranges set forth herein are exemplary only and are not intended to limit the scope or practice of the methods provided herein.

F. Diseases, Disorders and Therapeutic Products

The bifunctional molecules provided herein permit targeting of viral and bacterial vectors to cells that express targeted receptors. The targeted receptors are those that activate the PI3K signalling pathway and internalized linked ligands by virtue thereof. Because such receptors are diverse and widespread, the use of bifunctional molecules provides a flexible means for gene delivery and therapeutic product delivery to cells and tissues. Receptors for the targeting agents, such as growth factors and hormones, are overexpressed on cells associated with various disorders and conditions and/or on only selected cell types.

The use of the PI3K signaling strategy to tailor delivery of the adenovirus vectors to cell types over-expressing the desired growth factor receptor or cytokine receptor or other cell surface receptor that activates the PI3K pathway permits internalization via such receptors. Adenovirus binding and internalization can be further enhanced by taking advantage of additional interactions with CAR and also with integrins, if they are present on the cell surface. The bifunctional molecules provided herein do not bind the viral fiber protein, thus allowing for interactions with CAR. In addition, preferred bifunctional molecules bind the penton base protein. which has five “RGD” integrin binding sites. Since not all five “RGD” sites are bound by the whole molecule, the presence of the bifunctional molecule precludes neither CAR nor integrin interactions, and in addition offers a specific growth factor/cytokine—receptor binding interaction. In that sense, it is like a “triple-edged sword” for enhanced levels of binding and internalization, once targeting has been achieved. It is also advantageous to adopt a targeting strategy, which does not preclude interaction of the adenovirus with the αv integrins because the integrins are known to not only signal on their own, but to promote optimal activation of growth factor receptors (Vuori et al., Science, 266:1576 (1994); Cybulsky et al., J. Clin. Invest., 94:68 (1994); Jones et al., J. Cell Biol., 139:279 (1997); Miyamoto et al., J. Cell Biol., 135:1633 (1996); Schneller et al., EMBO J., 16:5600 (1997); Woodard et al., J. Cell Sci., 111:469 (1998); Moro et al., EMBO J., 17:6622 (1998); Soldi et al., EMBO J., 18:882 (1999)).

1. Diseases and Disorders

Methods for specifically targeting recombinant adenovirus vectors for delivery of gene products, particularly therapeutic products are provided herein. These methods are particularly suitable for targeting cells that express receptors to which the bifunctional molecules provided herein selectively bind resulting in internalization of the linked delivery vector. Adenoviruses are presently preferred. Diseases that can be targeted include, but are not limited to, cancers, vascular disorders, diabetic retinopathies, restenosis, ophthalmic disorders, hyperproliferative disorders and hormonal disorders. The methods and bifunctional molecules provided herein permit targeting to restricted sets of cells associated with particular disorders.

Angiogenesis

In the normal adult, angiogenesis is tightly regulated and limited to wound healing, pregnancy and uterine cycling. Angiogenesis is turned on by specific angiogenic molecules such as basic and acidic fibroblast growth factor (FGF), vascular endothelial growth factor (VEGF), angiogenin, transforming growth factor (TGF), tumor necrosis factor-α (TNF-α) and platelet derived growth factor (PDGF). Angiogenesis can be suppressed by inhibitory molecules such as interferon-α, thrombospondin-1, angiostatin and endostatin. It is the balance of these naturally occurring stimulators and inhibitors that is controls the normally quiescent capillary vasculature. When this balance is upset, as in certain disease states, capillary endothelial cells are induced to proliferate, migrate and ultimately differentiate.

Angiogenesis plays a central role in a variety of disease including cancer and neovascularization. Sustained growth and metastasis of a variety of tumors has also been shown to be dependent on the growth of new host blood vessels into the tumor in response to tumor derived angiogenic factors. Proliferation of new blood vessels in response to a variety of stimuli occurs as the dominant finding in the majority of eye disease and that blind including proliferative diabetic retinopathy (PDR), age-related macular degeneration (ARMD), rubeotic glaucoma, interstitial keratitis and retinopathy of prematurity. In these diseases, tissue damage can stimulate release of angiogenic factors resulting in capillary proliferation. VEGF plays a dominant role in iris neovascularization and neovascular retinopathies. While reports clearly show a correlation between intraocular VEGF levels and ischemic retinopathic ocular neovascularization, FGF likely plays a role. Basic and acidic FGF are known to be present in the normal adult retina, even though detectable levels are not consistently correlated with neovascularization. This may be largely due to the fact that FGF binds very tightly to charged components of the extracellular matrix and may not be readily available in a freely diffusible form that would be detected by standard assays of intraocular fluids.

A final common pathway in the angiogenic response involves integrin-mediated information exchange between a proliferating vascular endothelial cell and the extracellular matrix. This class of adhesion receptors, called integrins, are expressed as heterodimers having an a and β subunit on all cells. One such integrin, αᵥβ₃, is the most promiscuous member of this family and allows endothelial cells to interact with a wide variety of extracellular matrix components. Peptide and antibody antagonists of this integrin inhibit angiogenesis by selectively inducing apoptosis of the proliferating vascular endothelial cells Two cytokine-dependent pathways of angiogenesis exist and may be defined by their dependency on distinct vascular cell integrins, αᵥβ₃ and αᵥβ₅. Specifically, basic FGF- and VEGF-induced angiogenesis depend on integrin αᵥβ₃ and αᵥβ₅, respectively, since antibody antagonists of each integrin selectively block one of these angiogenic pathways in the rabbit corneal and chick chorioallantoic membrane (CAM) models. Peptide antagonists that block all αᵥ integrins inhibit FGF- and VEGF-stimulated angiogenesis. While normal human ocular blood vessels do not display either integrin, αᵥβ₃ and αᵥβ₅ integrins are selectively displayed on blood vessels in tissues from patients with active neovascular eye disease. While only αᵥβ₃ was consistently observed in tissue from patients with ARMD,αᵥβ₃ and αᵥβ₅ were present in tissues from patients with PDR. Systemically administered peptide antagonists of integrins blocked new blood vessel formation in a mouse model of retinal vasculogenesis.

In addition to adhesion events described above, cell migration through the extracellular matrix also depends on proteolysis. Matrix metalloproteinases are a family of zinc-requiring matrix-degrading enzymes that include the collagenases, gelatinases and stromelysins, all of which have been implicated in invasive cell behavior. Invasive cell processes such as tumor metastasis and angiogenesis have been found to be associated with the expression of integrins and MMP-2, MMP-2 are all found throughout the eye where they may interact to maintain a quiescent vasculature until the balance is upset, resulting in pathological angiogenesis. A non-catalytic C-terminal hemopexin-like domain of MMP-2 (PEX) can block cell surface collagenolytic activity and inhibit angiogenesis in the CAM model by preventing localization of MMP-2 to the surface of invasive cells through interaction with the integrin αᵥβ₃.

Anti-angiogenic agents have a role in treating retinal degeneration to prevent the damaging effects of trophic and growth factors. Angiogenic agents, also have role in promoting desirable vascularization to retard retinal degeneration by enhancing blood flow to cells.

Growth Factors/Cytokines and Pathological Conditions EGF Receptors

EGF receptors are overexpressed in glioblastomas, bladder tumors, advanced gastric tumors and cervical cancers (Gullick (1991) Br. Med. Bull., 47:87). They are overexpressed in 63% of tumor specimens from patients with lung cancer (Pastorino et al. (1993) J. Cell. Biochem. Suppl., 17F:237), and are overexpressed in astrocytic gliomas (Goussia et al. (2000) Oncol. Rep. 7:401. Overexpression is also correlated with tumor invasion and progression of human esophageal and gastric carcinomas (Yoshida et al., Exp. Pathol., 40:291 (1990)).

FGF Receptors

These receptors are overexpressed in human pancreatic adenocarcinomas (Kobrin et al., Cancer Res., 53:4741 (1993)), in human breast and gynecological cancers (Jaakkola et al., Int. J. Cancer, 54:378 (1993)), human astrocytomas (Morrison et al., J. Neuro-oncol., 18:207 (1994)); and in human melanoma tissues (Xerri et al., Melanoma Res., 6:223 (1996)). FGF receptor expressing cells are also implicated in restenosis, Kaposi sarcoma, diabetic retinopathies and numerous disorders of the eye.

EGFR, FGFR and IGF-1R

EGFR, FGFR and IGF-1R are overexpressed in pancreatic cancers (Korc, Surg. Oncol. Clin. N. Am., 7:25 (1998))

IGF-1R

IFG-1R is overexpressed and hyperphosphorylated in primary breast tumors (Surmacz, J. Mamm. Gland Biol. Neoplasia, 5:95 (2000)).

TNFR

Overexpression of both TNFα receptors, p55 and p75, is observed in neoplastic cells from patients with chronic lymphocytic leukemia (Waage et al., Blood, 80:2577 (1992)) TNFα p55 receptor is overexpressed in breast carcinomas (Pusztai et al., Br. J. Cancer, 70:289 (1994)). TNFR is overexpressed in normal and malignant myeloid cells, e.g., HL-60 promyelocytes (Munker et al., Blood, 70:1730 (1987)).

SCF Receptors

Development of mastocytosis (abnormal infiltration of mast cells into various organs) in patients with myelodysplastic syndrome (MDS) is due to a mutation in c-kit (the SCF receptor), which renders it ultra-sensitive to SCF (Dror et al., Br. J. Haematol., 108:729 (2000)). Therefore, SCF-derived bifunctional molecules can be used to deliver therapeutic products to such cells. Benign and malignant ovarian tumors express c-kit (SCF receptor) while normal tissue does not (Tonary et al., Int. J. Cancer, 89:242 (2000)).

2. Therapeutic Products

Among the DNA that encodes therapeutic products contemplated for use is DNA encoding correct copies of defective genes, such as the defective gene (CFTR) associated with cystic fibrosis (see, e.g., International Application WO 93/03709, which is based on U.S. Application Ser. No. 07/745,900; and Riordan et al. (1989) Science 245:1066-1073), and anticancer agents, such as tumor necrosis factors, and cytotoxic agents. Therapeutic products include but are not limited to, wild-type genes that are defective in targeted ocular disorders, such as defective gene products or fragments thereof sufficient to correct the genetic defect, trophic factors, including growth factors, inhibitors and agonists of trophic factors, anti-apoptosis factors and other products herein or known to those of skill in the art to be useful for treatment of selected disorders.

The following examples are included for illustrative purposes only and are not intended to limit the scope of the invention.

### EXAMPLE 1

Preparation and Characterization of DAV-1

A monoclonal antibody, designated DAV-1, had been previously obtained and characterized (see, Stewart et al. (1997) EMBO J. 16:1189-1198) as described in this Example. The nucleic acid and protein sequences of the heavy and light chain of DAV-1 are set forth in SEQ ID Nos. 1-4; and the nucleic acid and protein sequences of the portion used in exemplified fusion proteins containing substantially full-length heavy chain is set forth in SEQ ID Nos. 5 and 6.

Materials and Methods

Cell Lines, Viruses and Recombinant Proteins

A549, HeLa, H2981 and SW480 cell lines, adenovirus serotypes Ad2, Ad3 and Ad4 were purchased from the American Type Tissue Culture Collection (Rockville, Md.). For virus isolation, HeLa cells were infected with either Ad2, Ad3 or Ad4 at a multiplicity of 10 p.f.u./cell and then harvested 48-72 h later. Cells were frozen and thawed five times to release intracellular virus particles. After removing the cell debris by high-speed centrifugation, virions were isolated by banding on a 15-40% cesium chloride gradient in 10 mM Tris-HCl, 150 mM NaCl pH 8.1 (TBS) as reported previously (Everitt et al., 1977). Banded virions were removed and then dialyzed into TBS buffer containing 10% glycerol, except for cryo-EM studies in which case the CsCI was removed by multiple centrifugation steps in a Microcon 100 (Amicon) filtration device using pH 8.1 phosphate buffer. Recombinant Ad2 penton base containing 571 amino acids (Neumann et al., 1988) was produced in Sf9 insect cells using baculovirus as previously described (Nemerow et al., 1993); Wickham et al., 1993).

Generation and Characterization of the DAV-1 Anti-penton Base mAb

A hybridoma (designated DAV-1) secreting a mAb of the subtype ylK was generated by standard techniques. The DAV-1 mAb was purified from ascites fluids using protein G-Sepharose (HiTrap GII, Pharmacia). Fab fragments of the DAV-1 mAb were generated by papain digestion. Briefly, 1-5 mg/ml of purified DAV-1 lgG in 50 mM Tris-HCl pH 8.0, 10 mM I-cysteine, 3 mM EDTA was incubated for 7 h at 37° C. in the presence of 8% w/w soluble papain (Sigma Chemical Co., St. Louis, Mo.). The reaction was stopped by the addition of 30 mM iodoacetamide, and the Fab antibody fragments were then isolated on a Resource S FPLC column (Pharmacia) equilibrated with 50 mM MES pH 5.0. The purified Fab fragments were analyzed by SDS-PAGE and then concentrated to 2.2 mg/ml using a Centricon 10 membrane ultrafiltration device (Amicon).

Reactivity of the DAV-1 mAb with different adenovirus serotypes was quantified in an ELISA. Ninety-six well polystyrene plates (Immobilon, Dynatech) were coated with 1 μg of penton base or with 5 μg of purified Ad2, Ad3 or Ad4 in PBS for 18 h at 4° C. After blocking non-specific binding sites with 2% non-fat dried milk, 10 μg/ml of purified DAV-1 mAb or an irrelevant control antibody were added to the wells for 60 min at 22° C. Antibody binding was detected by the addition of alkaline phosphatase linked to goat anti-mouse IgG followed by substrate (Sigma Chemical Co., St. Louis, Mo). Substrate development was quantified at 405 nm in an ELISA plate reader (Titertek, Flow laboratories).

To examine whether the DAV-1 mAb also recognized RGD-containing cell matrix proteins, 1-2 μg of recombinant penton base protein, or fibronectin, vitronectin, collagen (type 1) and fibrogen were reacted with the DAV-1 mAb in a Western blot. Recombinant penton base or cell matrix proteins were electrophoresed on a 8-15% gradient SDS gel (Novex, San Diego, Calif.) and then transferred to a nitrocellulose filter (Immobilon P, Millipore). Following blocking of non-specific binding sites with 1% non-fat dried milk (Blotto), the filters were reacted with 10 μg/ml of the DAV-1 mAb followed by incubation with alkaline phosphatase linked to goat anti-mouse IgG (Tropix, Bedford, Mass.) and then with a chemiluminescent substrate (CDP).

Functional Analysis of the DAV-1 mAb

The effect of the DAV-1 mAb on penton base binding to cell surface αᵥ integrins was examined as follows. To 1×10⁶ A549 epithelial cells 10 μg/ml of purified DAV-1 IgG or Fab fragments of the DAV-1 mAb was added. Varying amounts of ¹²⁵I-labeled penton base (10,μCi/μg) were then added to the cells in the presence or absence of a 50-fold excess of unlabeled penton base and incubated for 60 min at 4° C. Unbound penton base was removed by centrifuging the cell samples through a cushion of 1:1 glycerol/mineral oil and the amount of cell-associated penton base was determined by counting the cell pellet in a γ-counter.

The effect of the DAV-1 mAb on adenovirus infection was quantified by plaque assay. A549 cells were seeded into six-well plates and cultured to 90% confluency. Three μg/ml of DAV-1 Fab or 18 μg of whole IgG DAV-1 antibody were added to the cell cultures, followed by addition of purified 100 p.f.u. Ad2 and incubation at 37° C. for 2 h. The Ad2 and antibody mixtures were then removed, and 8 ml of overlay medium containing 0.5% agarose in DMEM medium and 10% FCS was added into each well. The cells were fed with 4 ml of overlay medium on day 5 post-infection. The plaques were scored on day 10 post-infection. Epitope mapping and kinetic analysis of DAV-1 binding to penton base

As noted above, the DAV-1 binding site on the Ad2 penton base was identified by affinity-directed mass spectrometry. For these studies, a region of the penton base that approximately spanned the RGD-containing epitope sequence was selected. A series of overlapping synthetic peptides varying by one amino acid on the N-terminal or C-terminal region of the Ad2 penton base RGD sequence, ⁴⁸⁰MNDHAIRGDTFATRA⁴⁹⁴ (SEQ ID NO. 19), was generated by solid-phase protocols, and the precise boundaries of the DAV-1 epitope were then determined by affinity-directed mass spectrometry (see, Zhao et al. (1994) Anal. Chem. 66:3723-3726; Zhao et al. (1996) Proc. Natl. Acad. Sci. U.S.A. 93:4020-4024).

Precise measurements of DAV-1 interactions with the penton base protein were determined by SPR (Karlsson et al. (1991) J. Immunol. Methods 145:229-240) using an automated biosensor system (BIAcore 2000 Pharmacia). Briefly, recombinant penton base at 60 μg/ml was immobilized onto carboxymethyl dextran-coated biosensor chips in 10 mM MES pH 6.5 containing 10 mM NaCl. Following amine coupling of the penton base, varying amounts of purified Fab fragments (3.6-57.0 μg/ml) or IgG molecules (36-576 μg/ml) were flowed over the penton base at a rate of 40 μl/min, respectively. Kinetic binding data (Kₒₙ, K_{off} and K_{D}) were obtained using BlAevaluation software (version 2.1). Stoichiometric data was obtained by observing the change in SPR at saturation binding and assuming a molecular mass of 350 kDa for the penton base, 43 kDa for Fab fragments and 150 kDa for IgG molecules. The sequence recognized by DAV-1 was IRGDTFATR (see SEQ ID NO. 20).

### EXAMPLE 2

Preparation and Analysis of Bifunctional Signaling Antibodies

A. Cloning of DAV-1-encoding cDNA

The hybridoma secreting a monoclonal antibody of the type γ1κ (designated DAV-1) was generated as described in EXAMPLE 1 (see, Stewart et. al. (1997) EMBO J. 16:1189-1198). Total RNA was isolated from the DAV-1 hybridoma cell line (Trizol reagent, Gibco BRL) and cDNA was generated using the Superscript Plasmid System kit (Gibco BRL) essentially as described (Uematsu, Immunogenetics, 34: 174-178 (1991)). The DAV-1 heavy chain was PCR amplified with: primers 5′-CCT GCT CTG TGT TTA CAT GAG GG (CH3 region) (SEQ ID. NO. 15); 5′-CCC AGG GTC ATG GAG TTA G (CH1 region) (SEQ ID. NO. 16); for kappa (light) chain amplification: 5′-AAG ATG GAT ACA GTT GGT GC (CL-A) (SEQ ID. NO. 17) and 5′-TGT CAA GAG CTT CAA CAG GA (CL-B) (SEQ ID. NO. 18) were the primers used. PCR amplification was carried out using the parameters 94° C. for 1 minute, 63° C. for 1 minute and 72° C. for 1 minute, for a total of 30 cycles. The PCR products were ligated into pCR2.1 (Invitrogen, Carlsbad, Calif.) using a TA cloning kit (Invitrogen, Carlsbad, Calif.), and then sequenced by automated sequencing. Further amplification to obtain the complete DAV-1 heavy and light chains was performed using standard PCR reactions as described above.

The cDNA coding the γ heavy and κ-light chains of (DAV-1) were subcloned into an expression vector, designated plZ, which contains a Zeocin selection marker for expression in insect cells (Invitrogen, Calif.). The portion of the DAV-1 heavy chain used to generate fusion proteins with various cytokines or growth factors was the full-length heavy chain minus the last 18 amino acids (coding portion in SEQ ID NO. 1 encoding amino acid 1 through 438, and SEQ ID NO. 5) was cloned into the plZ expression vector between its KpnI and EcoRI restriction sites by introducing Kpnl and EcoRI sites into the 5′ and 3′ ends, respectively. (see, SEQ ID. NO. 5). This was accomplished by using the “sense” PCR primer 5′-GGT ACC GCC ACC ATG GGA TGG AGC TGG ATC T (SEQ ID. NO. 21) having a KpnI restriction site, and the “antisense” primer 5′-GAA TTC ATG TAA CAC AGA GCA GGA (SEQ ID. NO. 22) having an EcoRI restriction site, for PCR amplification of the portion of the DAV-1 heavy chain sequence set forth in SEQ ID. NO. 5, prior to cloning. The DAV-1 light chain (SEQ ID NO. 3) was cloned into the HindIII and Xbal sites of the plZ expression vector by introducing HindIII and Xbal sites into the 5′ and 3′ ends of the light chain encoding DNA (see SEQ ID. NO. 3). This was accomplished by using the “sense” PCR primer 5′-AAG CTT GCC ACC ATG GAG ACA GAC ACA ATC CTG CT (SEQ ID. NO. 23) having a HindIII restriction site, and the “antisense” primer 5′-TCT AGA TGT CTC TAA CAC TCA TTC CTG T (SEQ ID. NO. 24) having an Xbal restriction site, for PCR amplification of the DAV-1 light chain sequence set forth SEQ ID. NO. 3, prior to cloning. The resulting vectors were designated as plZ-γ, plZ-κ for heavy chain and light chain constructs, respectively.

Fab′₂ forms of the DAV-1 antibody were also PCR amplified by creating a C-terminal deletion in the DAV-1 γ-heavy chain at amino acid position 247 (see, SEQ ID No. 1), and were cloned between the Kpnl and EcoRI sites of the plZ vector using the “sense” primer sequence set forth in SEQ ID. NO. 21 (having a Kpnl restriction site), and the “antisense” primer 5′-GAA TTC TGA TAC TTC TGG GAC TGT (SEQ ID. NO. 25 with an an EcoRI restriction site).

B. Cloning of the Bifunctional Signaling Antibodies

DNA encoding full length mature human TNF-α, IGF-1, EGF and SCF peptides were obtained by PCR amplification of: cDNA obtained from ATCC (Rockville, Md.) encoding human TNF-α; RT-PCR of total RNA isolated from U937 cells for human IGF-1; cDNA obtained from Invitrogen, Carlsbad, Calif. for SCF; and a synthetic template prepared by annealing two oligonucleotides having an 18 bp overlap (SEQ ID NOS. 30 and 31) for EGF. For construction of the fusion proteins, DNA sequences encoding the full length mature human TNF-α, IGF-1 and EGF peptides (peptide sequences set forth in SEQ ID. NOS. 7, 8 and 9 respectively) were ligated in frame into the EcoRI site in plZ-γ downstream (3′-end) of the DAV-1 heavy chain portion (SEQ ID. NO. 5) used to prepare the fusion proteins.

To facilitate cloning of the full length mature peptides into the plZ-γ vector at the EcoRI site 3′ to the DAV-1 heavy chain sequence, EcoRI sites were introduced at the 5′ and 3′-ends of the nucleic acid encoding each of the full length mature peptides TNF-α, IGF-1 and EGF by PCR amplification using the following primers: For amplification of TNF-α, “sense” primer 5′-GAA TTC GTC AGA TCA TCT TCT CGA AC (SEQ ID. NO. 26) and “antisense” primer 5′-GAA TTC TAC AGG GCA ATG ATC CCA AA (SEQ ID. NO. 27); for amplification of IGF-1, “sense” primer 5′-GAA TTC GGA CCG GAG ACG CTC TGC GG (SEQ ID. NO. 28) and “antisense” primer 5′-GAA TTC TAA GCT GAC TTG GCA GGC TT (SEQ ID. NO. 29); for amplification of EGF, “sense” primer 5′-GAA TTC AAT AGT GAC TCT GAA TGT CCC CTG TCC CAC GAT GGG TAC TGC CTC CAT GAT GGT GTG TGC ATG TAT ATT GAA GCA TTG GAC AAG TAT GCA (SEQ ID. NO. 30) and “antisense” primer 5′-GAA TTC TAG CGC AGT TCC CAC CAC TTC AGG TCT CGG TAC TGA CAT CGC TCC CCG ATG TAG CCA ACA ACA CAG TTG CAT GCA TAC TTG TCC AAT GCT TC (SEQ ID. NO. 31). The orientation of the fusion proteins was determined by PCR analysis. All sequences were confirmed using automated DNA sequencing.

The fusion proteins contain the portion of the DAV-1 heavy chain sequence set forth in SEQ ID. NO. 6, followed by a two amino acid “linker” sequence generated by the EcoRI site between the DAV-1 and growth factor sequences (see amino acids 439 (Glu) and 440 (Phe) in SEQ ID NOS. 11, 12 and 13), followed in-frame by the full length, mature growth factor peptide. Sequences of fusion proteins of DAV-1 heavy chain with TNF-α, IGF-1 and EGF are set forth in SEQ ID. NOS. 11, 12 and 13, respectively.

For construction of the fusion protein of DAV-1 heavy chain with SCF, the DNA sequence encoding the full length mature SCF peptide (peptide sequence set forth in SEQ ID. NO. 10) was ligated in frame between the Notl and Xbal sites of the plZ-y vector, downstream (3′-end) of the DAV-1 heavy chain portion (SEQ ID. NO. 5) used to prepare the fusion protein. To facilitate cloning of the full length mature SCF peptide into the plZ-γ vector at the Notl and Xbal sites 3′ to the DAV-1 heavy chain sequence, Notl and Xbal sites were introduced at the 5′ and 3′-ends of the SCF sequence encoding the full length mature peptide by PCR amplification using the following primers: “sense” primer 5′-GCG GCC GCA AGG GAT CTG CAG GAA TCG (SEQ ID. NO. 32) and “antisense” primer 5′-TCT AGA GTG CAA CAG GGG GTA ACA TA (SEQ ID. NO. 33). Generation of the Noti site at the 5′-end of the full length SCF-encoding nucleic acid resulted in change of the first amino acid from glutamic acid (Glu) in the wildtype sequence (see first amino acid of SEQ ID. NO> 10) to glutamine (Gln) in the fusion protein (see amino acid 450 in SEQ ID. NO. 14). The resulting fusion construct includes the portion of the DAV-1 heavy chain peptide sequence set forth in SEQ ID. NO. 6, followed by 11 amino acids of “linker” sequence (amino acids 439 to 449 in SEQ ID. NO. 14), followed by the SCF mature peptide. The sequence of the resulting fusion protein is set forth in SEQ ID. NO. 14.

Similar cloning strategies were employed to generate growth factors such as TNF-α, IGF-1, EGF and SCF fused to the 3′-end of the Fab′₂ forms of the DAV-1 antibody.

C. Generation of Secreted Bifunctional Antibody Fusion Proteins

The DAV-1 heavy chain-TNF, heavy chain-IF-1 or heavy chain-EGF expression vector and the DAV-1 light chain expression vector were co-transfected into SF9 insect cells. The SF9 insect cells (Invitrogen) were transfected with a total of 3 μg plasmid DNA comprising the heavy chain-growth factor/cytokine fusion and light chain vectors using 15 μl Superfect (Qiagen) in 200 μl DMEM (serum-free) at room temperature for 15 minutes and then added to fresh cultures of SF9 cell monolayers (about 90% confluency). Transfected cells were then subjected to Zeocin selection (600 μg/ml) for the production of bifunctional molecules using a penton base ELISA assay. A pool of positive Zeocin-resistant cells was then selected for the production of fusion proteins.

D. Purification and Functional Analysis of Bifunctional Molecules

Supernatants from transfected SF9 cells were assayed for bifunctional antibody production by an ELISA assay using immobilized penton base as previously described (Mathias et al., J. Virol., 72(11): 8669 (1998)) and as described in this Example. Recombinant Ad2 penton base was produced in Trichoplusia Tn 5B1-4 insect cells (Wickham et al., Biotech. Prog., 8:391-396 (1992)) using the baculovirus vector pBlueBac (Invitrogen, Carlsbad, Calif.). The recombinant Ad2 penton base was used to coat a 96-well plastic tissue culture plate (Immulon-4, Dynatech) at a concentration of 1 μg protein/well. Non-specific binding sites were quenched by incubation with a blocking agent (Superblock; Pierce), and then the SF9 cell culture supernatants were added to the plates and incubated at 22° C. for 1 h. Bound antibody was detected with HRP conjugated rabbit anti-mouse antibody (whole molecule). Culture supernatants that tested positive for the bifunctional antibody were passed through a Protein L (Actigen, Cambridge, UK) affinity column. Bound antibody was eluted with either 50 mM diethylamine (pH9.7) or 0.1 M citrate-0.15 M NaCl (pH 3.0), and pooled fractions were dialyzed against PBS (10 mM sodium phospate, 150 nM NaCl, pH 7.2). The purified fusion proteins were further characterized by SDS-PAGE and western blot.

The cytotoxic activity of the DT bifunctional monoclonal antibodies (DAV-1-TNFα bifunctional antibody) resulting from its interaction with TNF-α receptors was assayed using the TNF-sensitive MCF-7 cell line as previously described (Xiang et al., J. Biotech., 53: 3 (1997).

SF9 cell supernatants or the purified bifunctional proteins were separated on 12% SDS-PAGE gels and either stained with Coomassie blue or transferred to PVDF membrane filters (Amersham) and probed with rabbit anti-mouse (Sigma, St. Louis, Mo.) or goat anti-human TNF-α (Chemicon, Temecula, Calif.) antibodies followed by secondary antibodies conjugated to horseradish peroxidase (Sigma, St. Louis, Mo.) and detection with a chemiluminescence reagent (Supersignal, Pierce, Rockford, Ill.). Western blot analyses showed that the DAV-1 monoclonal antibody (D molecule) and the DAV-1-TNFα bifunctional antibody (DT molecule) were both recognized by an anti-mouse IgG antibody, while only the DT molecule was recognized by an anti-TNFα polyclonal antibody.

### EXAMPLE 3

Gene Delivery Vectors and Complexing with Bifunctional Molecules

A. Adenovirus Propagation

Cell lines that are commonly used for growing adenovirus are useful as host cells for the preparation of adenovirus packaging cell lines. Preferred cells include 293 cells, an adenovirus-transformed human embryonic kidney cell line obtained from the ATCC, having Accession Number CRL 1573; HeLa, a human epithelial carcinoma cell line (ATCC Accession Number CCL-2); A549, a human lung carcinoma cell line (ATCC Accession Number CCL 1889); and other epithelial-derived cell lines. As a result of the adenovirus transformation, the 293 cells contain the E1 early region regulatory gene. All cells were maintained in complete DMEM+10% fetal calf serum unless otherwise noted.

These cell lines allow the production and propagation of adenovirus-based gene delivery vectors that have deletions in preselected gene regions and that are obtained by cellular complementation of adenoviral genes. Such units include but are not limited to E1 early region, E4 and the viral fiber gene.

Adenovirus type 2 (Ad2, ATCC) was propagated in A549 (ATCC # CCL 185) epithelial cells and purified as previously described (Wickham et al., Cell, 73: 309 (1993)) and as described in this Example. Cells were infected with Ad2 at a multiplicity of infection (MOI) of 10 and then harvested 2-3 days later. Cells were frozen and thawed five times to release intracellular particles, and then the cell debris was removed by centrifugation. The cell lysate was subjected to density gradient-ultracentrifugation on 25%-40% cesium chloride gradients, and the virus band was removed and dialyzed against 40 mM Tris-HCl-buffered saline, pH 8.1, containing 10% glycerol.

B. Adenovirus Gene Delivery Vectors

Adenoviral vectors for delivery of genes, such as therapeutic genes, and methods for their construction and propagation are well known and readily available (see, e.g., co-pending U.S. application Ser. No. 09/482,682; International PCT application No. PCT/US00/00265, and U.S. application Ser. No. 09/562,934). Other delivery vectors may be used. A variety of Ad delivery vectors are known and available.

In general, exemplary fiber-expressing and fiberiess recombinant adenovirus vectors have been described (Von Seggern et al., J. Virol., 73: 1601 (1999); copending U.S. application Ser. No. 09/482,682 filed Jan. 14, 2000, and also International PCT application No. PCT/US00/00265, filed Jan. 14, 2000)). Construction of Ad5.βgal.wt and Ad5.μgal.ΔF (deposited on Jan. 15, 1999, the ATCC under accession number VR2636) is described therein. Ad2 or Ad5 vectors using the RSV LTR in place of the SV40 promoter can be constructed in a manner analogous to the similar Ad5-based vectors described in the above-noted applications. Such vectors were used in the experiments in the Examples herein. As described therein, gutted Ad vectors are those from which most or all viral genes have been deleted. They are grown by co-infection of the producing cells with a “helper” virus (using an E1-deleted Ad vector). The helper virus transcomplements the missing Ad functions, including production of the viral structural proteins needed for particle assembly. The helper virus can be a fiber-deleted Ad (such as that described in Von Seggern et al., J. Virol. 73:1601-1608 (1999)). The vector is prepared in a fiber expressing cell line (see, e.g., Von Seggern et al. (1998) J. Gen. Virol. 79:1461-1468; Von Seggern et al. (2000), J. Virol. 74:354-362). All the necessary Ad proteins except fiber are provided by the fiber-deleted helper virus, and the particles are equipped with the particular fiber expressed by the host cells.

A helper adenovirus vector genome and a gutless adenoviral vector genome are delivered to a packaging cell line (see, e.g., International PCT application No. PCT/US00/00265, filed Jan. 14, 2000). The cells are maintained under standard cell maintenance or growth conditions, whereby the helper vector genome and the packaging cell together provide the complementing proteins for the packaging of the adenoviral vector particle. Such gutless adenoviral vector particles are recovered by standard techniques. The helper vector genome may be delivered in the form of a plasmid or similar construct by standard transfection techniques, or it may be delivered through infection by a viral particle containing the genome. Such viral particle is commonly called a helper virus. Similarly, the gutless adenoviral vector genome may be delivered to the cell by transfection or viral infection.

The helper virus genome is preferably a fiberless adenovirus vector genome. Preferably, such genome also lacks the genes encoding the adenovirus E1A and E1B proteins. More preferably, the genome further lacks the adenovirus genes encoding the adenovirus E3 proteins. Alternatively, the genes encoding such proteins may be present but mutated so that they do not encode functional E1A, E1 B and E3 proteins. Furthermore, such vector genome may not encode other functional early proteins, such as E2A, E2B, and E4 proteins. Alternatively, the genes encoding such other early proteins may be present but mutated so that they do not encode functional proteins.

The packaging cell also provides proteins necessary for the complementation of the gutless vector so that an adenovirus particle containing the gutless vector genome may be produced. Thus, the packaging cell line can provide wild-type or modified fiber protein. Alternatively, the cell line could package a fiberless particle, which could be used by itself or to which exogenously provided fiber could be added.

In producing gutless vectors, the helper virus genome is also packaged, thereby producing helper virus. In order the minimize the amount of helper virus produced and maximize the amount of gutless vector particles produced, it is preferable to delete or otherwise modify the packaging sequence in the helper virus genome, so that packaging of the genome is prevented or limited. Since the gutless vector genome will have a packaging sequence, it will be preferentially packaged. One way to do this is to mutate the packaging sequence by deleting one or more of the nucleotides comprising the sequence or otherwise mutating the sequence to inactivate or hamper the packaging function. An alternative approach is to engineer the helper genome so that recombinase target sites flank the packaging sequence and to provide a recombinase in the packaging cell. The action of recombinase on such sites results in the removal of the packaging sequence from the helper virus genome. Preferably, the recombinase is provided by a nucleotide sequence in the packaging cell that encodes the recombinase. Most preferably, such sequence is stably integrated into the genome of the packaging cell. Various kinds of recombinase are known by those skilled in the art. The preferred recombinase is Cre recombinase, which operates on so-called lox sites, which are engineered on either side of the packaging sequence as discussed above. Further information about the use of Cre-loxP recombination is found in U.S. Pat. No. 5,919,676 and Morsy and Caskey, Molecular Medicine Today, January 1999, pgs. 18-24.

As the gutless vectors lack many or all Ad genes, they must be grown as mixed cultures in the presence of a helper virus which can provide the missing functions. To date, such helper viruses have provided all Ad functions except E1, and E1 is complemented by growth in 293 cells or the equivalent. The resulting virus particles are harvested, and the helper virus is typically removed by CsCl gradient centrifugation (the vector chromosome is generally shorter than the helper chromosome, resulting in a difference in buoyant density between the two particles).

An example of a gutless gene delivery vector is pAdARSVDys (Haecker et al. (1996) Human Gene Therapy 7:1907-1914). This plasmid contains a full-length human dystrophin cDNA driven by the RSV promoter and flanked by Ad inverted terminal repeats and packaging signals. Desired therapeutic proteins and other products intended for delivery to cells can be readily substituted for the dystrophin gene in this vector.

293 cells are infected with a first-generation Ad which serves as a helper virus, and then transfected with purified vector DNA. The helper Ad genome and the delivery vector DNA are replicated as Ad chromosomes, and packaged into particles using the viral proteins produced by the helper virus. Particles are isolated and the delivery vector-containing particles separated from the helper by virtue of their smaller genome size and therefore different density on CsCl gradients.

The vector is grown in either 633 or 705 cells and Ad5.βgal.ΔF is used as a helper virus; both helper and the delivery vector genomes replicate and are packaged into particles. The provides all the essential Ad proteins except fiber, and the fiber protein is that produced by the cells (Ad5 fiber in 633 cells and Ad37 fiber in the case of 705 cells). The packaged viral particles are then isolated by centrifugation.

For experiments exemplified herein, the following first generation recombinant virus and its fiberless derivative were used. The first-generation Ad2 virus Ad.RSV.β-gal is an E-1 and E-3 deleted, replication-defective, recombinant vector containing a Rous Sarcoma virus regulatory sequence—driven LacZ reporter gene (Stratford-Perricaudet et al., J. Clin. Invest., 90:626 (1992)).

First, a recombinant plasmid pAd.RSVβ-gal was constructed, in which the LacZ gene with the SV40 early region polyadenylation signal driven by the Rous Sarcoma virus long terminal repeat (RSV-LTR) is inserted downstream of the 1.3 map units (mu) from the left end of the adenovirus type 5 (Ad5) genome in place of E1a and E1b (mu 1.3-9.4). The reporter gene is followed by mu 9.4-17 of Ad5 to allow homologous recombination with the adenoviral genome for the generation of the recombinant adenovirus. The recombinant adenovirus was constructed by in vivo homologous recombination between plasmid pAd.RSVβ-gal and Ad5 dl327, an E-3 deletion mutant of Ad5 (Trousdale, M. D. et al., Cornea, 14:280 (1995)).

Briefly, cells were cotransfected with 5 μg of linearized pAd.RSVβ-gal and 5 μg of the 2.6-100 mu fragment of Ad5 DNA. After overlaying with agar and incubation for 10 days at 37° C., plaques containing recombinant adenovirus were picked and screened for nuclear β-galactosidase activity. The Ad.RSVβ-gal/ΔF vector is identical to the first-generation virus with the exception of a fiber deletion to generate fiberless virions.

As noted, these and other exemplary fiber-expressing and fiberless recombinant adenovirus vectors have been described (Von Seggern et al., J. Virol., 73: 1601 (1999); copending U.S. application Ser. No. 09/482,682 filed Jan. 14, 2000, and also International PCT application No. PCT/US00/00265, filed Jan. 14, 2000)).

C. Complexing of Adenovirus Delivery Vector Particles with Bifunctional Molecules

Complexation of the recombinant adenovirus vectors with the bifunctional molecules was accomplished as follows: The E1 deleted adenovirus vector encoding LacZ, under control of the RSV LTR, in place of E1 (Ad.RSV.β-gal vector described above), was incubated with DAV-TNF (DT molecules) or control DAV (D molecules) antibodies at a ratio of 2 antibody molecules per RGD motif of adenovirus at room temperature for 30 minutes in either EXEL-400 medium (JRH Bioscience, Lenexa, Kans.) or Dulbecco modified Eagle's medium (DMEM) (Gibco-BRL).

### EXAMPLE 4

Adenovirus, Gene Express, Cell Binding and Internalization Assays

A. Gene Delivery and Expression Assay

The complex of Example 3C was then mixed with M21-L12 human melanoma cells, which are deficient in αᵥβ₃ and αᵥβ₅ integrin (Wickham et al., Cell, 73: 309 (1993)), on ice for 60 min. Unbound virus was removed by washing with ice-cold PBS, and the cells were warmed to 37° C. for varying times and then plated in tissue culture plates. Ad-mediated gene transfer was examined 24 or 48 hours post infection by staining for β-galactosidase activity as previously described, by incubating the cells for 60 min at 37° C. with 3.5 mM o-nitrophenyl-β-D-galactopyranoside (ONPG) as a chromagenic substrate and in buffer containing 0.5% Nonidet P-40 (Li et al., J. Virol., 72: 2055 (1998); Huang et al., J. Virol., 70: 4502 (1996)).

B. Binding Assay

To measure adenovirus binding to cells, 500 μg aliquots of adenovirus vector was labeled with ¹²⁵I by incubation with lodogen (Pierce) in an lodogen-coated tube containing 1 mCi of Na¹²⁵I. Iodinated proteins were separated from free ¹²⁵I by gel filtration as described (Huang et al. (1999) J. Virol. 73:2798-2802). Binding of radiolabeled adenovirus on M21-L12 cells was then quantitated as described earlier (Huang et al. (1999) J. Virol. 73:2798-2802), and as described in this Example. 106 cells in suspension were incubated with 10⁶ cpm (viral particles were generally labeled to a specific activity of 5×10⁶ to 8×10⁶ cpm/μg) of the labeled virus proteins at 4° C. for 2 h. Non-specific binding was determined by incubating cells and labeled proteins in the presence of a 100-fold excess of unlabeled virus or fiber protein. After the cells were washed four times in ice-cold phosphate buffered saline, specific binding was calculated by subtracting the non-specific binding from the total bound cpm.

C. Internalization Assay

To measure virus endocytosis, cells were incubated with complexed ¹²⁵I-labeled adenovirus and DAV-TNF or control antibodies, in DMEM supplemented with 0.5% purified bovine serum albumin (BSA) at 4° C. for 60 minutes. After removal of non-bound virus by washing with ice-cold PBS, the cells were warmed to 37° C. for varying times. Uninternalized virions were removed by incubation with trypsin-EDTA at room temperature for 5 min. prior to counting the cell pellets.

### EXAMPLE 5

Adenovirus complexed with bifunctional molecules targeted to receptors that promote internalization of ligands by PI3K activation are internalized via binding to the targeted receptors.

Analysis of Bifunctional Signaling Antibodies

DAV-1 bifunctional signaling antibodies, designated DT (DAV-1 fused to TNF-α), DI (DAV-1 fused to IGF-1), and DE (DAV-1 fused to EGF), were expressed in insect cells as secreted proteins and purified on Protein L affinity columns. The DT heavy chain (D_{H}T) had an apparent molecular weight of approximately 70 kDa, consistent with the combined sizes of the DAV-1γ heavy chain (53 kDa) and monomeric TNF-α ligand (17 kDA). The apparent molecular weight of the κ light chains of DAV-1 (D_{L}) was identical to that of the recombinant DT molecule (approx. 25 kDa). Western blot analyses showed that the DAV-1 mAb (D) and the DT molecules were recognized by an anti-mouse IgG antibody, while only the DT molecule was recognized by an anti-TNF-α polyclonal antibody.

DT molecules were capable of binding to immobilized penton base or Ad particles in an ELISA and elicited cytotoxicity against a TNF-α sensitive cell line, MCF-7, indicating that the DT bifunctional molecule retains virus and cytokine receptor binding functions.

Bifunctional Molecules Promote Ad-mediated Gene Delivery to αv Integrins

A first generation adenovirus vector containing a RSV-driven LacZ reporter gene with DT was preincubated at a ratio of 2 antibody molecules per RGD motif. This complex was then added to M21-L12 human melanoma cells, which do not express αv integrins (Felding-Habermann et al. (1992) J. Clin. Invest. 89:2018-2022), but can support efficient virus binding (Wickham et al. (1993) Cell 73:309-319). Ad complexed with DT but not D alone, significantly increases Ad-mediated gene delivery to M21-L12 cells as measured by transgene expression at 48 hrs post-infection. Approximately 60% of cells incubated with Ad plus DT stained positive for β-galactosidase, compared to less than 3% of cells that had been incubated with virus alone or virus plus D. The increase in gene delivery by DT was not due to increased activation of the RSV LTR transgene promoter as a consequence of ligation of the TNF receptor, since M21-L12 cells that had been infected with adenovirus alone for three hours followed by addition of DT showed very little increase in gene delivery at 48 hours post-infection. This result indicates that bifunctional molecules increase adenovirus-mediated gene delivery by enhancing one or more steps associated with cell entry.

DT Molecules Enhance Ad Binding and IIternalization

The following experiments showed that DT enhancement of gene delivery was associated with increased virus attachment to M21-L12 cells. ¹²⁵I-labeled Ad particles alone or complexed with D or DT molecules were analyzed for binding to M21-L12 cells. Binding of ¹²⁵I-Ad complexed with DT molecules was also examined in the absence or presence of an excess of recombinant Ad5 fiber protein, a polyclonal anti-human TNF-α antibody or a combination of the TNF-α antibody and fiber protein prior to addition to M21-L12 cells. ¹²⁵I-labeled Ad was incubated with DT or D molecules prior to addition to M21-L12 cells and incubation on ice for 60 min. Unbound Ad was removed by washing and the cells were warmed to 37° C. for varying times to allow virus internalization as measured by resistance to trypsinization. The results of these experiments showed that pre-incubation of ¹²⁵I-labeled Ad particles with DT but not with D molecules increased virus binding approximately 5-fold.

To investigate the molecules responsible for increased binding, competition experiments were performed. Ad-DT binding to cells was measured in the presence of a 50-fold excess of recombinant fiber protein or anti-TNF-α or a combination of these molecules. Either recombinant fiber or anti-TNF-α antibody alone was capable of blocking only 20-25% of Ad-DT binding to cells. In contrast, approximately 70% of binding could be inhibited by a combination of fiber and anti-TNF-α. These findings indicate that Ad-DT binding to cells is mediated by CAR-fiber internalization as well as TNF-α-receptor association.

DT Molecules Potentiate Internalization of ¹²⁵I-labeled Virus Particles as Measured by Resistance to Trypsin Digestion

As demonstrated in earlier studies (Wickham et al. (1993) Cell 73:309-319), because M21-L12 cells do not express αv integrins, relatively low levels of adenovirus internalization by these cells occurs. DT molecules significantly increased the rate and extent of adenovirus internalization into these cells. These findings indicate that DT molecules enhance gene delivery by promoting virus binding as well as virus internalization.

DT Enhancement of Gene Delivery is Associated with PI3K Activation

Efficient Ad internalization via αv integrins requires activation of PI3K, a key cellular signaling molecule. Experiments demonstrating that DT enhancement of gene delivery is mediated by PI3-kinase were performed. In these experiments, to show that DT enhancement of gene delivery also involves PI3K, M21-L12 melanoma cells were pretreated with the PI3K inhibitors wortmannin (30 nM) or LY292004 (20 μM) for 30 min prior to the addition of Ad.RSV.βgal complexed with D or DT molecules. Ad mediated gene delivery was analyzed 48 hours post infection. The results show that wortmannin and LY294002 inhibited Ad-mediated gene delivery by approximately 70% and 50%, respectively, indicating that PI3K activity plays a major role in DT enhancement or gene delivery.

For further demonstration of the role of PI3K-dependent signaling in enhanced gene delivery, Ad-mediated gene delivery by other bifunctional molecules whose cytokine/growth factor domains are known to activate PI3K was measured. Bifunctional molecules that target PI3K signaling pathways (DT=DAV-1-TNF; DE=DAV1-EGF; DI=DAV-1-IF-1) also enhance Ad-mediated gene delivery to M21-L12 cells. DT, DE and DI molecules enhanced gene delivery by approximately 30, 10 and 5 fold respectively. Enhanced gene delivery by these molecules was also inhibited by pretreatment of cells with wortmannin. These findings further demonstrate that PI3K activation promotes Ad gene delivery.

### EXAMPLE 6

Demonstration of Targeted Delivery Via Growth Factor Receptors

Bifunctional Molecules Allow Gene Delivery by Fiberless Adenovirus Particles

Fiberless adenovirus vector that cannot bind to CAR have been constructed (see Example 3 and also, e.g., copending U.S. application Ser. No. 09/482,682 and U.S. application Ser. No. 09/562,934; see, also Von Seggern et al. (1999) J.Virol. 73:1601-1608). The structure of these particles is nearly identical to that of wildtype virions. Fiberless particles alone showed almost no transgene delivery to SW480 epithelial cells, even though these cells express CAR and integrin αvβ5 (Von Seggern et al. (1999) J. Virol. 73:1601-1608). To show that bifunctional molecules promote gene delivery by a fiberless Ad vector, CAR and αv integrin-expressing SW480 cells were infected with a fiberless adenovirus or fiberless virus complexed with DT molecules at a ratio of 1 antibody molecule per viral particle. The reporter gene expression was examined by β-gal staining 48 hours post infection. The fiberless virus was incubated with bifunctional molecules before incubation with SW480 cells at 37° for 15 min. Ad-mediated gene delivery was examined 48 post infection.

The results demonstrated that DT molecules enhanced gene delivery of fiberless viruses. Fiberless particles complexed with DT or DI molecules exhibited increased gene delivery approximately 10-15, 3 and 5 fold, respectively, compared to the uncomplexed fiberless particles. These findings indicate that fiberless particles can be retargeted to cells via signal transducing antibodies.

Since modifications will be apparent to those of skill in this art, it is intended that this invention be limited only by the scope of the appended claims.

1. A bifunctional molecule, comprising:
an antibody or antigen-binding portion thereof and a targeting agent, wherein:
the antibody specifically binds to an antigen in a protein that binds to αᵥ integrin; and
the targeting agent specifically binds to a cell surface protein that activates the phosphatidylinositol 3 (PI3K) signaling pathway.

2. The bifunctional molecule of claim 1, wherein the targeting agent or portion thereof triggers phosphatidylinositol-3-OH kinase (PI3K) activation.

3. The bifunctional molecule of claim 1, further comprising a linker that links the antibody or antigen-binding portion thereof to the targeting agent.

4. The bifunctional molecule of claim 1 that comprises a fusion protein.

5. The bifunctional molecule of claim 1 that comprises chemically conjugated polypeptides.

6. The bifunctional molecule of claim 2, wherein the linker is a single amino acid or a peptide.

7. The bifunctional molecule of claim 1, wherein the antibody comprises a heavy chain or a portion thereof sufficient for antigen-binding fused to the targeting agent.

8. The bifunctional molecule of claim 1, wherein the antibody portion an Fab′2 fragment.

9. The bifunctional molecule of claim 1, wherein the antibody portion comprises a sufficient portion of the variable regions of the heavy and light chains for antigen recognition.

10. The bifunctional molecule of claim 1, wherein the antibody comprises the sequence of amino acids set forth in SEQ ID No. 2 or SEQ ID No. 6 or a sufficient portion thereof for antigen recognition.

11. The bifunctional molecule of claim 1, wherein the antibody comprises the sequence of amino acids set forth in SEQ ID No. 4 or a sufficient portion thereof for antigen recognition.

12. The bifunctional molecule of claim 1, wherein the antibody portion is an Fab fragment.

13. The bifunctional molecule of claim 10, wherein the nucleic acid encoding the antibody portion selected from
(a) the coding portion of the sequence of nucleotides set forth in SEQ ID No. 1 or SEQ ID No.5;
(b) a sequence of nucleotides that comprises one or more degenerate codons of (a); and
(c) a sequence of nucleotides that hybridizes along its full length under conditions of high stringency to a sufficient portion of (a) or (b) to encode an antigen-binding portion of the antibody.

14. The bifunctional molecule of claim 11, wherein the nucleic acid encoding the antibody portion is selected from
(a) the coding portion of the sequence of nucleotides set forth in SEQ ID No. 3;
(b) a sequence of nucleotides that comprises one or more degenerate codons of (a); and
(c) a sequence of nucleotides that hybridizes along its full length under conditions of high stringency to a sufficient portion of (a) or (b) to encode an antigen-binding portion of the antibody.

15. The bifunctional molecule of claim 1, comprising the sequence of amino acids set forth in any of SEQ ID Nos. 7-14 for specific binding to a targeted receptor.

16. The bifunctional molecule of claim 1, wherein the protein that binds to αᵥ integrin is a viral protein or a bacterial protein that interacts with αᵥ integrins for internalization of the respective virus or bacteria.

17. The bifunctional molecule of claim 16, wherein the virus is selected from adenovirus and adeno-associated virus.

18. The bifunctional molecule of claim 1, wherein the antibody or portion thereof specifically binds to the penton base of an adenovirus.

19. The bifunctional molecule of claim 1, wherein the antibody or portion thereof specifically binds to an antigen that includes an RGD motif.

20. The bifunctional molecule of claim 1, wherein the targeting agent comprises all or sufficient portion thereof of a protein that binds to G-protein coupled receptors, oncogene product receptors, hormone receptors or cytokine receptors that employ the PI3 signalling pathway for signal transduction,
wherein the sufficient portion thereof specifically binds to the cell surface receptor therefor and internalizes linked viral or bacterial particles.

21. The bifunctional molecule of claim 1, wherein the targeting agent comprises all or sufficient portion thereof of a protein that binds to G-protein coupled receptors that employ the PI3 signalling pathway for signal transduction,
wherein the sufficient portion thereof specifically binds to the cell surface receptor therefor and internalizes linked viral or bacterial particles.

22. The bifunctional molecule of claim 1, wherein the targeting agent comprises all or sufficient portion thereof of hormone or growth factor or cytokine,
wherein the sufficient portion thereof is specifically bind to the cell surface receptor therefor and internalizes linked viral or bacterial particles.

23. The bifunctional molecule of claim 6, wherein the targeting agent or portion thereof is a tumor necrosis factor (TNF), an fibroblast growth factor (FGF), an insulin-like growth factor (IF) a colony stimulating factor (CSF), insulin or a serum cell factor (SCF).

24. The bifunctional molecule of claim 6, wherein the targeting agent or portion thereof is insulin, IF-1, TNF-α, SCF, CSF, a PDGF, an FGF, a heparin binding epidermal growth factor (HEGF), a VEGF or dimer thereof.

25. The bifunctional molecule of claim 6, wherein the targeting agent or portion thereof is TNF-α, IF-1, SCF or EGF.

26. The bifunctional molecule of claim 1, wherein targeted cell surface protein is selected from among a PDGF receptor, an IF-1 receptor, an EGF receptor, a member of the FGF receptor family, a TNF receptor, a CSF-1 receptor, an insulin receptor, an IF-1 receptor, an NGF receptor, an II-2 receptor, an II-3 receptor, an II-4 receptor, an IgM receptor, a CD4 receptor, a CD2 receptor, a CD3/T cell receptor, a G protein linked thrombin receptor, an ATP receptor, and an fMLP receptor.

27. The bifunctional molecule of claim 1, wherein the targeted cell surface protein is selected from among tyrosine kinase receptors that, when activated, result in increased accumulation of Ptdlns(3,4,5)P3, receptors associated with the src family non-receptor tyrosine kinases that stimulate PI3Ks phosphorylate phosphatidylinositol(3,4,5)P3 (Ptdlns(3,4,5)P3) accumulation.

28. An isolated nucleic acid molecule(s), comprising a sequence of nucleotides that encodes the bifunctional molecule of claim 1.

29. A targeted delivery vector, comprising:
a bifunctional molecule of claim 1; and
a viral or bacterial vector.

30. The targeted delivery vector of claim 29, wherein the gene delivery vector encodes a therapeutic product.

31. The targeted delivery vector of claim 29, wherein the vector is an adenovirus vector.

32. The targeted delivery vector of claim 29, wherein the vector is a fiberless adenovirus vector.

33. The targeted delivery vector of claim 29, wherein the bifunctional molecule and viral or bacterial vector are complexed by interaction of the antibody portion of the bifunctional molecule with a viral or bacterial surface protein.

34. The targeted delivery vector of claim 12, wherein the bifunctional molecule and viral or bacterial vector wherein the antibody portion of the bifunctional molecule is covalently linked to the viral or bacterial surface protein.

35. An isolated nucleic acid molecule, comprising as sequence of nucleotides encoding the bifunctional molecule of claim 1.

36. A combination, comprising:
a delivery vector for delivering gene products to targeted cells; and
a bifunctional molecule of claim 1.

37. The combination of claim 36, wherein the bifunctional molecule and delivery vector for delivering gene products to targeted cells are complexed.

38. A method of targeted gene therapy, comprising administering a combination of claim 36.

39. A method of targeted gene therapy, comprising administering a combination of claim 37.


SEQUENCE LISTING (33 sequences)

SEQ ID NO: 1 - DNA, 1516 bp, Mouse
  CDS (28)...(1395): DAV-1 heavy chain, penton base monoclonal antibody
cagacactgaacacactgactctaaccatgggatggagctggatctttctcttcctcctgtcaggaactgcaggcgtccactctgaggtccagcttcagcagtcaggacctgagctggtgaaacctggggcctcagtgaagatatcctgcaaggcttctggatacacattcactgactacaacatgcactgggtgaagcagagccatggaaagagccttgagtggattggatatatttatccttacaaaggtggtactggctacaaccagaagttcaagagcaaggccacattgacaacagacagttcctccaacacagcctacatggagctccgcagcctgacatctgatgcctctgcagtctattactgtgcaagagggattgcttactggggccaagggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcatctgctctgtgttacatgagggcctgcacaaccaccatactgagaagagcctctcccactctcctggtaaatgatcccagtgtccttggagccctctggtcctacaggactctgtcacctacctccacccctccctgtataaataaagcacctagcactgccttgggaccctgcaataaaaaaaaaaaaaaa

SEQ ID NO: 2 - protein, 456 aa, Mouse
  PEPTIDE (0)...(0): DAV-1 heavy chain, penton base monoclonal antibody
MGWSWIFLFLLSGTAGVHSEVQLQQSGPELVKPGASVKISCKASGYTFTDYNMHWVKQSHGKSLEWIGYIYPYKGGTGYNQKFKSKATLTTDSSSNTAYMELRSLTSDASAVYYCARGIAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFICSVLHEGLHNHHTEKSLSHSPGK

SEQ ID NO: 3 - DNA, 831 bp, Mouse
  CDS (13)...(726): DAV-1 light chain, penton base monoclonal antibody
aagcttaccgccatggagacagacacaatcctgctatgggtgctgctgctctgggttccaggctccactggtgacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatttagaatctgggatcccagccaggtttagtggcagtgggtctgggacagacttcaccctcaacatccatcctgtggaggaggaggatgctgcaacctattactgtcagcaaactaatgaggatccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttagagacaaaggtcctgagacgccaccaccagctccccagctccatcctatcttcccttctaaggtcttggaggcttcctcgagcggtaaagggcgaattccagc

SEQ ID NO: 4 - protein, 238 aa, Mouse
  PEPTIDE (0)...(0): DAV-1 light chain, penton base monoclonal antibody
METDTILLWVLLLWVPGSTGDIVLTQSPASLAVSLGQRATISCKASQSVDYDGDSYMNWYQQKPGQPPKLLIYAASNLESGIPARFSGSGSGTDFTLNIHPVEEEDAATYYCQQTNEDPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 5 - DNA, 1314 bp, Mouse
  CDS (0)...(1314): Portion of DAV-1 heavy chain used for fusion protein bifunctional antibody
atgggatggagctggatctttctcttcctcctgtcaggaactgcaggcgtccactctgaggtccagcttcagcagtcaggacctgagctggtgaaacctggggcctcagtgaagatatcctgcaaggcttctggatacacattcactgactacaacatgcactgggtgaagcagagccatggaaagagccttgagtggattggatatatttatccttacaaaggtggtactggctacaaccagaagttcaagagcaaggccacattgacaacagacagttcctccaacacagcctacatggagctccgcagcctgacatctgatgcctctgcagtctattactgtgcaagagggattgcttactggggccaagggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcatctgctctgtgttacat

SEQ ID NO: 6 - protein, 438 aa, Mouse
  PEPTIDE (0)...(0): Portion of DAV-1 heavy chain used for fusion protein bifunctional antibody
MGWSWIFLFLLSGTAGVHSEVQLQQSGPELVKPGASVKISCKASGYTFTDYNMHWVKQSHGKSLEWIGYIYPYKGGTGYNQKFKSKATLTTDSSSNTAYMELRSLTSDASAVYYCARGIAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFICSVLH

SEQ ID NO: 7 - protein, 157 aa, Human [sequence omitted]

SEQ ID NO: 8 - protein, 70 aa, Human
  PEPTIDE (0)...(0): Human Insulin-like Growth Factor 1 sequence (IGF-1, mature peptide)
GPETLCGAELVDALQFVCGDRGFYFNKPTGYGSSSRRAPQTGIVDECCFRSCDLRRLEMYCAPLKPAKSA

SEQ ID NO: 9 - protein, 53 aa, Human
  PEPTIDE (0)...(0): Epidermal Growth Factor (EGF, mature peptide)
NSDSECPLSHDGYCLHDGVCMYIEALDKYACNCVVGYIGERCQYRDLKWWELR

SEQ ID NO: 10 - protein, 164 aa, Human
  PEPTIDE (0)...(0): Stem Cell Factor (SCF, mature peptide)
EGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVA

SEQ ID NO: 11 - protein, 597 aa, Artificial Sequence
  Fusion protein with N-terminal portion of DAV-1 heavy chain and TNF alpha mature peptide
MGWSWIFLFLLSGTAGVHSEVQLQQSGPELVKPGASVKISCKASGYTFTDYNMHWVKQSHGKSLEWIGYIYPYKGGTGYNQKFKSKATLTTDSSSNTAYMELRSLTSDASAVYYCARGIAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFICSVLHEFVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 12 - protein, 510 aa, Artificial Sequence
  Fusion protein with N-terminal portion of DAV-1 heavy chain and IGF-1 mature peptide
MGWSWIFLFLLSGTAGVHSEVQLQQSGPELVKPGASVKISCKASGYTFTDYNMHWVKQSHGKSLEWIGYIYPYKGGTGYNQKFKSKATLTTDSSSNTAYMELRSLTSDASAVYYCARGIAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFICSVLHEFGPETLCGAELVDALQFVCGDRGFYFNKPTGYGSSSRRAPQTGIVDECCFRSCDLRRLEMYCAPLKPAKSA

SEQ ID NO: 13 - protein, 493 aa, Artificial Sequence
  Fusion protein with N-terminal portion of DAV-1 heavy chain and EGF mature peptide
MGWSWIFLFLLSGTAGVHSEVQLQQSGPELVKPGASVKISCKASGYTFTDYNMHWVKQSHGKSLEWIGYIYPYKGGTGYNQKFKSKATLTTDSSSNTAYMELRSLTSDASAVYYCARGIAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFICSVLHEFNSDSECPLSHDGYCLHDGVCMYIEALDKYACNCVVGYIGERCQYRDLKWWELR

SEQ ID NO: 14 - protein, 613 aa, Artificial Sequence
  Fusion protein with N-terminal portion of DAV-1 heavy chain and SCF mature peptide
MGWSWIFLFLLSGTAGVHSEVQLQQSGPELVKPGASVKISCKASGYTFTDYNMHWVKQSHGKSLEWIGYIYPYKGGTGYNQKFKSKATLTTDSSSNTAYMELRSLTSDASAVYYCARGIAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFICSVLHEFCRYPAQWRPQGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVA

SEQ ID NO: 15 - DNA, 23 bp, Artificial Sequence
  PCR primer for amplification of CH3 region of DAV-1 heavy chain.
cctgctctgtgtttacatgaggg

SEQ ID NO: 16 - DNA, 19 bp, Artificial Sequence
  PCR primer for amplification of CH1 region of DAV-1 heavy chain.
cccagggtcatggagttag

SEQ ID NO: 17 - DNA, 20 bp, Artificial Sequence
  PCR primer for amplification of DAV-1 kappa chain CL-A.
aagatggatacagttggtgc

SEQ ID NO: 18 - DNA, 20 bp, Artificial Sequence
  PCR primer for amplification of DAV-1 kappa chain CL-B.
tgtcaagagcttcaacagga

SEQ ID NO: 19 - protein, 15 aa, Adenovirus
  PEPTIDE (0)...(0): Peptide spanning integrin binding site on penton base.
MNDHAIRGDTFATRA

SEQ ID NO: 20 - protein, 9 aa, Adenovirus
  PEPTIDE (0)...(0): Epitope on penton base integrin binding site recognized by DAV-1.
IRGDTFATR

SEQ ID NO: 21 - DNA, 31 bp, Artificial Sequence
  PCR sense primer for subcloning DAV-1 heavy chain for whole antibody or Fab+402 constructs.
ggtaccgccaccatgggatggagctggatct

SEQ ID NO: 22 - DNA, 24 bp, Artificial Sequence
  PCR antisense primer for subcloning DAV-1 heavy chain for whole antibody construct.
gaattcatgtaacacagagcagga

SEQ ID NO: 23 - DNA, 35 bp, Artificial Sequence
  PCR sense primer for subcloning DAV-1 light chain for whole antibody or Fab+402 constructs.
aagcttgccaccatggagacagacacaatcctgct

SEQ ID NO: 24 - DNA, 28 bp, Artificial Sequence
  PCR antisense primer for subcloning DAV-1 light chain for whole antibody or Fab+402 constructs.
tctagatgtctctaacactcattcctgt

SEQ ID NO: 25 - DNA, 24 bp, Artificial Sequence
  PCR antisense primer for subcloning DAV-1 heavy chain for Fab′2 constructs.
gaattctgatacttctgggactgt

SEQ ID NO: 26 - DNA, 26 bp, Artificial Sequence
  PCR sense primer for subcloning TNF. into DAV-1 /TNF. fusion construct.
gaattcgtcagatcatcttctcgaac

SEQ ID NO: 27 - DNA, 26 bp, Artificial Sequence
  PCR antisense primer for subcloning TNF. into DAV-1/TNF. fusion construct.
gaattctacagggcaatgatcccaaa

SEQ ID NO: 28 - DNA, 26 bp, Artificial Sequence
  PCR sense primer for subcloning IGF-1 into DAV-1/IGF-1 fusion construct.
gaattcggaccggagacgctctgcgg

SEQ ID NO: 29 - DNA, 26 bp, Artificial Sequence
  PCR antisense primer for subcloning IGF-1 into DAV-1/IGF-1 fusion construct.
gaattctaagctgacttggcaggctt

SEQ ID NO: 30 - DNA, 96 bp, Artificial Sequence
  PCR sense primer for subcloning EGF into DAV-1/EGF fusion construct.
gaattcaatagtgactctgaatgtcccctgtcccacgatgggtactgcctccatgatggtgtgtgcatgtatattgaagcattggacaagtatgca

SEQ ID NO: 31 - DNA, 98 bp, Artificial Sequence
  PCR antisense primer for subcloning EGF into DAV-1/EGF fusion construct.
gaattctagcgcagttcccaccacttcaggtctcggtactgacatcgctccccgatgtagccaacaacacagttgcatgcatacttgtccaatgcttc

SEQ ID NO: 32 - DNA, 27 bp, Artificial Sequence
  PCR sense primer for subcloning SCF into DAV-1/ SCF fusion construct.
gcggccgcaagggatctgcaggaatcg

SEQ ID NO: 33 - DNA, 26 bp, Artificial Sequence
  PCR antisense primer for subcloning SCF into DAV-1/SCF fusion construct.
tctagagtgcaacagggggtaacata